(19) United States
(12) Patent Application Publication
(10) Pub. No.: US 2023/0192895 A1
(43) Pub. Date: Jun. 22, 2023

(54) PHARMACOKINETIC & PHARMACODYNAMIC MODEL FOR DETERMINING EFFECTIVE DOSE OF ANTI-TICAGRELOR ANTIBODY

(71) Applicant: PhaseBio Pharmaceuticals, Inc., Malvern, PA (US)

(72) Inventor: John LEE, Malvern, PA (US)

(21) Appl. No.: 17/739,491

(22) Filed: May 9, 2022

Related U.S. Application Data
(60) Provisional application No. 63/186,515, filed on May 10, 2021.

(51) Int. Cl. C07K 16/44

(52) U.S. Cl. CPC C07K 16/44; A61K 2039/505

(57) ABSTRACT

The present disclosure provides a model to mathematically explore and predict the pharmacokinetic and pharmacodynamic relationships between of PB2452, its interaction with ticagrelor and the active metabolite, and the ability of PB2452 to reverse the antiplatelet effects of ticagrelor. This model may be used to determine dosing regiments of PB2452 for a particular patient population.

### CROSS REFERENCE TO RELATED APPLICATIONS

This application claims benefit of U.S. provisional application No. 63/186,515, which was filed on May 10, 2021, which is incorporated herein by reference in its entirety.

### REFERENCE TO SEQUENCE LISTING

The instant application contains a Sequence Listing, which has been submitted electronically in ASCII format via EFS-Web and is hereby incorporated by reference in its entirety. Said ASCII copy, created Dec. 16, 2022, is named PHAS-043_01US_SubSeqList_ST25.txt and is 44,957 bytes in size.

### FIELD OF THE INVENTION

The present disclosure provides a model to mathematically explore and predict the pharmacokinetic and pharmacodynamic relationships between of PB2452, its interaction with ticagrelor and the active metabolite, and the ability of PB2452 to reverse the antiplatelet effects of ticagrelor. This model may be used to determine dosing regiments of PB2452 for a particular patient population.

### BACKGROUND OF THE INVENTION

Antiplatelet therapy is an essential part of secondary prevention of cardiovascular events (Bhatt, Hulot, Moliterno, & Harrington, 2014). In particular, dual antiplatelet therapy—the combination of aspirin with an oral P2Y₁₂ receptor antagonist—is the predominant approach in patients with acute coronary syndromes, coronary-artery stenting, or previous myocardial infarction. Ticagrelor is a direct acting, reversibly binding, oral P2Y12 receptor antagonist (Teng, Maya, & Butler, 2013). The 180 mg loading dose followed by 90 mg twice daily, in combination with low dose aspirin, is used for the prevention of cardiovascular (CV) death, myocardial infarction (MI) and stroke in patients with acute coronary syndromes (ACS), based on the results of the PLATelet inhibition and patient Outcomes (PLATO) study (Wallentin, et al., 2009)

A major limitation of oral P2Y₁₂ receptor antagonists is the increased bleeding risk due to platelet suppression during treatment which persists for several days after drug cessation. The antiplatelet effects can markedly inhibit achievement of hemostasis in patients with spontaneous or procedure-related major bleeding. If an urgent or emergency procedure is indicated, the proceduralist must decide whether to proceed while accepting the increased bleeding risk or whether to postpone the procedure for several days and accept the increased ischemic risk after discontinuing the antiplatelet therapy and the risk associated with delaying a medically indicated procedure. The American College of Cardiology Foundation—American Heart Association, European Society of Cardiology, and other society guidelines recommend cessation of oral P2Y12 receptor antagonists for 3 to 7 days before surgery (Hillis, Smith, Anderson, & et al., 2011) (Valgimigli, Bueno, Byrne, & et al., 2018).

### SUMMARY OF THE INVENTION

The present disclosure provides methods of determining a dose and/or dosing regimen of an antibody or fragment thereof that binds to an inhibitor of P2Y12 signaling to restore P2Y12 receptor activity.

In some aspects, the present disclosure provides methods of modeling, simulating, and/or determining an effective dosing regimen of an antibody or fragment thereof that binds an inhibitor of P2Y₁₂ receptor signaling or P2Y₁₂ receptor-induced platelet aggregation in a patient population, the method comprising: a) determining a pharmacokinetic-pharmacodynamic (PD/PD) model that characterizes the relationship between ticagrelor and ticagrelor active metabolite (TAM) individually versus P2Y₁₂ receptor-induced platelet aggregation and P2Y₁₂ receptor signaling; b) wherein if the determination in (a) indicates that the P2Y₁₂ receptor-induced platelet aggregation and P2Y₁₂ receptor signaling is decreasing, the predicted effective dosing regimen is a higher dose of the antibody or fragment thereof infused at a faster rate or over a longer period of time; or c) wherein if the determination in (a) indicates that the P2Y₁₂ receptor-induced platelet aggregation and P2Y₁₂ receptor signaling is increasing, the predicted effective dosing regimen is a lower dose of the antibody or fragment thereof and/or infused at a slower rate or over a shorter period of time.

In some embodiments, the dosing regimen is sufficient to increase P2Y₁₂ receptor-induced platelet aggregation and P2Y₁₂ receptor signaling values towards the baseline observed before administration of the inhibitor of the P2Y₁₂ receptor signaling. In some embodiments, the dosing regimen is effective to sustain the increase of P2Y₁₂ receptor-induced platelet aggregation and P2Y₁₂ receptor signaling.

In some embodiments, P2Y₁₂ receptor-induced platelet aggregation and/or P2Y₁₂ receptor signaling is determined by one or more methods selected from light transmittance aggregometry (LTA), VerifyNow™-based P2Y₁₂ reactivity units (PRU), vasodilatory stimulated phosphoprotein (VASP) phosphorylation, and/or other platelet-function or P2Y₁₂-receptor-signaling assays.

In some embodiments, the metabolism of ticagrelor to TAM is modeled as a function of the concentration values of the antibody or fragment thereof.

In some embodiments, the pharmacokinetic-pharmacodynamic (PK/PD) model that characterizes the relationship between ticagrelor and ticagrelor active metabolite (TAM) individually versus P2Y₁₂ receptor-induced platelet aggregation or LTA and or P2Y₁₂ receptor signaling is determined using the following equation:

$$PRU = Base * ( 1 - \frac{E {max}_{1} * {TICA}^{γ}}{EC {50}_{1}^{γ} + {TICA}^{γ}} - \frac{E {max}_{2} * {TAM}^{γ}}{EC {50}_{2}^{γ} + {TAM}^{γ}} )$$

In some embodiments, the predicted effective dosing regimen comprises an initial bolus followed by a higher rate infusion, and then followed by a slower rate infusion.

In some embodiments, the values of P2Y₁₂ receptor-induced platelet aggregation and P2Y12 receptor signaling necessary for the intended patient population are maintained.

In some embodiments, the P2Y₁₂ receptor-induced platelet aggregation and P2Y₁₂ receptor signaling levels are maintained for about 1 to 48 hours. In some embodiments, the P2Y₁₂ receptor-induced platelet aggregation and P2Y₁₂ receptor signaling levels are maintained for about 10-30 hours. In some embodiments, the P2Y₁₂ receptor-induced platelet aggregation and P2Y₁₂ receptor signaling levels are maintained for about 20-24 hours.

In some embodiments, the dosing regimen provides complete reversal of the inhibitor of a P2Y₁₂ receptor-induced platelet aggregation and P2Y₁₂ receptor signaling. In some embodiments, the dosing regimen provides complete reversal of the inhibitor of a P2Y₁₂ receptor-induced platelet aggregation and P2Y₁₂ receptor signaling within about 5 minutes of infusion onset. In some embodiments, the complete reversal is sustained for at least 20 to 24 hours.

In some embodiments, administration of the antibody or fragment thereof restores platelet function. In some embodiments, administration of the antibody or fragment thereof restores platelet aggregation or platelet receptor signaling. In some embodiments, administration of the antibody or fragment thereof restores platelet aggregation or platelet receptor signaling to at least 80% of baseline. In some embodiments, administration of the antibody or fragment thereof restores platelet aggregation or platelet receptor signaling within 1 minute to 60 minutes of administration. In some embodiments, administration of the antibody or fragment thereof restores platelet aggregation or platelet receptor signaling within 5 minutes of administration.

In some embodiments, administration of the antibody or fragment thereof provides a sustained restoration of platelet aggregation or platelet receptor signaling. In some embodiments, the restoration of platelet aggregation or platelet receptor signaling is sustained for at least 12 hours after administration. In some embodiments, the restoration of platelet aggregation or platelet receptor signaling is sustained for at least 16 hours after administration. In some embodiments, the restoration of platelet aggregation or platelet receptor signaling is sustained for at least 24 hours after administration. In some embodiments, the antibody or fragment thereof is a Fab and the patient is administered a dose between about 1 g and about 48 g. In some embodiments, the dose is between about 9 g to about 18 g of the Fab. In some embodiments, the patient is administered a dose of about 1 g, about 3 g, about 9 g, about 18 g, about 24 g, about 30 g, about 36 g or about 48 g of the Fab. In some embodiments, the antibody or fragment thereof is administered to the patient intravenously. In some embodiments, the antibody or fragment thereof is administered intravenously over about 15 minutes to about 36 hours.

In some embodiments, the pharmaceutical composition is administered in two or more segments. In some embodiments, the first segment is a bolus. In some embodiments, the administration rates for each of the segments differ. In some embodiments, the administration rates for each of the segments differ for successive segments of the infusion. In some embodiments, the antibody or fragment thereof is administered in three or more segments, wherein the administration rates for each of the segments differ for successive segments of the infusion. In some embodiments, the predicted effective dosing regimen of the antibody or fragment thereof comprises a 6 gram IV bolus followed by 6 grams infused over 4 hours, and then 6 grams over 12 hours. In some embodiments, the antibody or fragment thereof is in a pharmaceutical composition comprising about 50 mg/mL to about 200 mg/mL of the anti-ticagrelor antibody or fragment thereof, about 5 mM to about 50 mM histidine/histidine hydrochloride buffer, about 100 mM to about 300 mM sucrose, and about 0.01% (w/v) to about 1.0% (w/v) polysorbate 80, pH 5.5 to 6.5 In some embodiments, the pharmaceutical formulation comprises 100 mg/mL of the anti-ticagrelor antibody or fragment thereof, 25 mM histidine/histidine hydrochloride buffer, 290 mM sucrose, and 0.05% (w/v) polysorbate 80, pH 6.0. the pharmaceutical formulation is diluted in saline for administration.

In some embodiments, the inhibitor of a P2Y₁₂ receptor signaling is ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-ydroxyethoxy)cyclopentane-1,2-diol) or a metabolite or derivative thereof.

In some embodiments, the antibody or a fragment thereof comprises complementarity-determining region (CDR) combinations selected from the group consisting of:

a) SEQ ID NO:53 (VH CDR1), SEQ ID NO:54 (VH CDR2), SEQ ID NO:55 (VH CDR3), SEQ ID NO:58 (VL CDR1), SEQ ID NO:59 (VL CDR2), and SEQ ID NO:60 (VL CDR3);

b) SEQ ID NO:63 (VH CDR1), SEQ ID NO:64 (VH CDR2), SEQ ID NO:65 (VH CDR3), SEQ ID NO:68 (VL CDR1), SEQ ID NO:69 (VL CDR2), and SEQ ID NO:70 (VL CDR3); and

c) SEQ ID NO:73 (VH CDR1), SEQ ID NO:74 (VH CDR2), SEQ ID NO:75 (VH CDR3), SEQ ID NO:78 (VL CDR1), SEQ ID NO:79 (VL CDR2), and SEQ ID NO:80 (VL CDR3).

In some embodiments, the antibody or a fragment thereof comprises a combination of heavy chain variable region (VH) and light chain variable region (VL) sequences selected from the group consisting of SEQ ID NO:52 and SEQ ID NO:57; SEQ ID NO:62 and SEQ ID NO:67; and SEQ ID NO:72 and SEQ ID NO:77.

In some aspects, the present disclosure provides methods of treating a patient using the predicted effective dosing regimen determine using the methods disclosed herein. In some embodiments, the patient has been administered ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-ydroxyethoxy)cyclopentane-1,2-diol). In some embodiments, the patient is experiencing or at risk of experiencing ticagrelor-associated bleeding. In some embodiments, the bleeding is major bleeding. In some embodiments, the bleeding is characterized by being major bleeding or life-threatening bleeding, potentially leading to clinically significant disability, requiring surgery to control the bleeding, requiring treatment with blood products, or is acute bleeding associated with a clinically important drop in hemoglobin.

In some embodiments, the patient requires urgent surgery or intervention. In some embodiments, the urgent surgery or intervention is known to be associated with a significant risk of bleeding, such as coronary artery bypass surgery, has an adverse surgical outcome if bleeding is not carefully controlled, such as neurological, ophthalmologic, or joint replacement surgery, is associated with risk of experiencing perioperative events; or is indicated in a patient at high risk of thrombosis if dual antiplatelet therapy is withheld perioperatively. In some embodiments, the patient is at risk of developing, or has been diagnosed with Acute Coronary Syndrome (ACS). In some embodiments, wherein the patient is at risk of developing, or has been diagnosed with a disease selected from the group consisting of myocardial infarction (MI), unstable angina, stable ischemic heart disease, in sickle cell disease, including pediatric patients, atrial fibrillation, coronary arterial disease, peripheral arterial disease, ischemic stroke, one or more coronary stents, carotid artery stents, stents following an intracranial aneurysm, and arterio-venous fistulae created for hemodialysis.

In some embodiments, the patient is a pediatric patient. In some embodiments, the pediatric patient is younger than 18 years old. In some embodiments, the pediatric patient is younger than 2 years old.

In some embodiments, the patient is an adult patient. In some embodiments, the adult patient is between 18 and 64 years old inclusive. In some embodiments, the patient is over 65 years old. In some embodiments, the patient is between 65 and 80 years old inclusive.

In some embodiments, the patient has been administered aspirin (acetylsalicylic acid).

### BRIEF DESCRIPTION OF THE FIGURES

FIG. 1 shows a schematic illustration of the combined ticagrelor, metabolite, and PB2452 pharmacokinetic model. Inputs into the system for ticagrelor and PB2452 are depicted with dashed lines. The model consists of two-compartment models (central and peripheral) for ticagrelor, metabolite, and PB2452. Ticagrelor is metabolized to form the active metabolite at a rate of Ktmet. PB2452 binds to ticagrelor and the metabolite to form complexes denoted as PB2452-TICA and PB2452-TAM respectively. These complexes dissociate to return ticagrelor, metabolite, and PB2452 to systemic circulation. The model assumes that the clearance for the complexes and PB2452 alone are the same. Additional PB2452-TICA and PB2452-TAM compartments were added to account for the process whereby ticagrelor and the metabolite are returned to circulation at a later timepoint. PB2452 is removed from the system. When the complexes dissociate from the original compartments, PB2452 does not return to systemic circulation in this model (represented by an X on the arrows).

FIG. 2 shows simulation results for PRU with the observed data overlaid by dosing regimen of PB2452. The solid line depicts the median of the simulation while the dashed lines are the 5ₜₕ and 95ₜₕ percentiles. The dotted line depicts the median baseline (prior to administration of TICA) of PRU for the cohort. The gray box reflects the time from the start of the first administration of PB2452 to the end of the last administration of PB2452.

FIG. 3 shows observed versus Individual Prediction plots for various measured components of the model.

FIG. 4 shows simulation results for various measured components of the model. The solid line depicts the median of the simulation while the dashed lines are the 5th and 95th percentiles. The dosing regimen for PB2452 was 6 g for 15 minutes followed by 6 g for 4 hours followed by 6 g for 12 hours.

### DETAILED DESCRIPTION

PB2452, a neutralizing monoclonal antibody fragment that binds the antiplatelet drug ticagrelor with high affinity, is being developed as a ticagrelor reversal agent. To identify a clinically useful intravenous (IV) reversal regimen, a semi-mechanistic exposure-response model was developed during the PB2452 first in human Phase 1 study.

In a randomized, double-blind, placebo-controlled, single-dose trial to evaluate the safety, efficacy, and PK of PB2452 in healthy volunteers pretreated with ticagrelor, sequential dose cohort data was used to build and refine an exposure-response model that combined population PK models for TICA, TAM, and PB2452, and related their binding relationships to the PK of uncomplexed TICA and TAM which is predictive of platelet inhibition in Emax models. Platelet function was assessed by multiple assays. The model was developed using Bayesian methods in NONMEM.

Human PK and PD data from sequential dose cohorts were used to initially define and then refine model parameters. Model simulations indicated that an initial IV bolus of PB2452, followed by a high-rate infusion, and then a slower-rate infusion would provide immediate and sustained reversal of the antiplatelet effects of ticagrelor. Based on model predictions, a 6 gram (g) IV bolus followed by 6 g infused over 4 h and then 6 g over 12 h was identified and tested in study subjects and shown to provide complete reversal within 5 minutes of infusion onset that was sustained for 20-24 h.

A semi-mechanistic exposure-response model of PB2452 reversal of ticagrelor was developed using Phase 1 first in human study data. Diagnostic plots, including visual predictive checks, show the model was predictive of the reversal profile of PB2452 and will inform future trials of PB2452.

### Ticagrelor

Ticagrelor works by binding to the P2Y₁₂ receptor on platelets, thereby preventing adenosine diphosphate, or ADP, from causing platelet aggregation. Ticagrelor binds transiently to the P2Y₁₂ receptor, cycling on and off, allowing anti-ticagrelor agents, such as PB2452, a human Fab fragment that binds to ticagrelor, to bind to free ticagrelor, thereby preventing ticagrelor's activation of the receptor and removing ticagrelor from circulation. With ticagrelor bound to PB2452 or removed, ADP can once again bind the P2Y₁₂ receptor and induce platelet aggregation.

### Anti-Ticagrelor Agents

In some aspects, the present disclosure provides agents that bind to ticagrelor ((1S,2S,3R,5S)-3-[7-{[(1R,2S)-2-(3,4-difluorophenyl)cyclopropyl]amino}-5-(propylthio)-3H-[1,2,3]-triazolo[4,5-d]pyrimidin-3-yl]-5-(2-ydroxyethoxy)cyclopentane-1,2-diol) or a metabolite or derivative thereof.

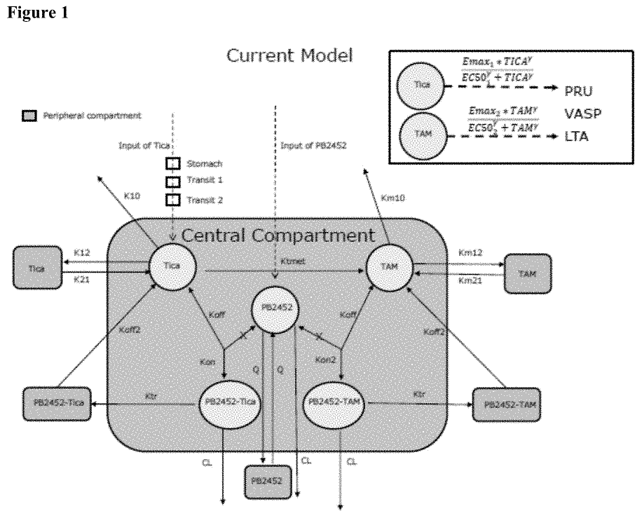

In some embodiments, the ticagrelor and/or metabolite thereof is depicted in FIG. 1. In some embodiments, the ticagrelor metabolite is an active metabolite.

In some aspects, the agent that binds to ticagrelor and/or a metabolite thereof is an antibody or a fragment thereof. In some embodiments, the antibody or fragment thereof is selected from, but is not limited to, a polyclonal antibody, a monoclonal antibody, a humanized antibody, a human antibody, a single chain Fv (scFv), a single domain antibody, a Fab, a F(ab′)2, a single chain diabody, an antibody mimetic, an antibody variable domain, a camelid antibody (also known as VHH or nanobody). In some embodiments, the antibody comprises a scFv. In some embodiments, the antibody or a fragment thereof comprises a Fab. In some embodiments the antibody mimetic is an adnectin molecule, an affibody molecule, an affilin molecule, an affimer molecule, an affitin molecule, an alphabody molecule, an anticalin molecule, an aptamer molecule, an armadillo repeat protein molecule, an atrimer molecule, an avimer molecule, a designed ankyrin repeat protein molecule (DARPin) molecule, a fynomer molecule, a knottin molecule, a knottin molecule, a Kunitz domain inhibitor molecule, a monobody, a nanoCLAMP molecule, or a nanofitin molecule.

In further embodiments of the above aspects and embodiments, the antibody or a fragment thereof comprises a heavy chain variable region (VH) sequence selected from the group consisting of SEQ ID NO:2, SEQ ID NO:12, SEQ ID NO:22, SEQ ID NO:32, SEQ ID NO:42, SEQ ID NO:52, SEQ ID NO:62, and SEQ ID NO:72; and a light chain variable region (VL) sequence selected from the group consisting of SEQ ID NO:7, SEQ ID NO:17, SEQ ID NO:27, SEQ ID NO:37, SEQ ID NO:47, SEQ ID NO:57, SEQ ID NO:67, and SEQ ID NO:77. In some embodiments, the antibody comprises a combination of VH and VL sequences selected from the group consisting of SEQ ID NO:2 and SEQ ID NO:7; SEQ ID NO:12 and SEQ ID NO:17; SEQ ID NO:22 and SEQ ID NO:27; SEQ ID NO:32 and SEQ ID NO:37; SEQ ID NO:42 and SEQ ID NO:47; SEQ ID NO:52 and SEQ ID NO:57; SEQ ID NO:62 and SEQ ID NO:67; and SEQ ID NO:72 and SEQ ID NO:77. In further embodiments, the antibody comprises a combination of VH and VL selected from the group consisting of SEQ ID NO:52 and SEQ ID NO:57; SEQ ID NO:62 and SEQ ID NO:67; and SEQ ID NO:72 and SEQ ID NO:77.

In further embodiments of the above aspects and embodiments, the antibody or a fragment thereof comprises framework regions (FR) and complementarity-determining regions (CDRs) 1, 2, and 3 of a heavy chain variable region and a light chain variable region, wherein the CDR1, CDR2, and CDR3 sequences of the heavy chain variable region comprise, SEQ ID NO:3 (CDR1), SEQ ID NO:4 (CDR2), and SEQ ID NO:5 (CDR3); SEQ ID NO:13 (CDR1), SEQ ID NO:14 (CDR2), and SEQ ID NO:15 (CDR3); SEQ ID NO:23 (CDR1), SEQ ID NO:24 (CDR2), and SEQ ID NO:25 (CDR3); SEQ ID NO:33 (CDR1), SEQ ID NO:34 (CDR2), and SEQ ID NO:35 (CDR3); SEQ ID NO:43 (CDR1), SEQ ID NO:44 (CDR2), and SEQ ID NO:45 (CDR3); SEQ ID NO:53 (CDR1), SEQ ID NO:54 (CDR2), and SEQ ID NO:55 (CDR3); SEQ ID NO:63 (CDR1), SEQ ID NO:64 (CDR2), and SEQ ID NO:65 (CDR3); or SEQ ID NO:73 (CDR1), SEQ ID NO:74 (CDR2), and SEQ ID NO:75 (CDR3); and wherein the CDR1, CDR2, and CDR3 sequences of the light chain variable region comprise, SEQ ID NO:8 (CDR1), SEQ ID NO:9 (CDR2), and SEQ ID NO:10 (CDR3); SEQ ID NO:18 (CDR1), SEQ ID NO:19 (CDR2), and SEQ ID NO:20 (CDR3); SEQ ID NO:28 (CDR1), SEQ ID NO:29 (CDR2), and SEQ ID NO:30 (CDR3); SEQ ID NO:38 (CDR1), SEQ ID NO:39 (CDR2), and SEQ ID NO:40 (CDR3); SEQ ID NO:48 (CDR1), SEQ ID NO:49 (CDR2), and SEQ ID NO:50 (CDR3); SEQ ID NO:58 (CDR1), SEQ ID NO:59 (CDR2), and SEQ ID NO:60 (CDR3); SEQ ID NO:68 (CDR1), SEQ ID NO:69 (CDR2), and SEQ ID NO:70 (CDR3); or SEQ ID NO:78 (CDR1), SEQ ID NO:79 (CDR2), and SEQ ID NO:80 (CDR3). In further embodiments, the antibody comprises a combination of CDR regions selected from the group consisting of: SEQ ID NO:53 (VH CDR1), SEQ ID NO:54 (VH CDR2), SEQ ID NO:55 (VH CDR3), SEQ ID NO:58 (VL CDR1), SEQ ID NO:59 (VL CDR2), and SEQ ID NO:60 (VL CDR3); SEQ ID NO:63 (VH CDR1), SEQ ID NO:64 (VH CDR2), SEQ ID NO:65 (VH CDR3), SEQ ID NO:68 (VL CDR1), SEQ ID NO:69 (VL CDR2), and SEQ ID NO:70 (VL CDR3); and SEQ ID NO:73 (VH CDR1), SEQ ID NO:74 (VH CDR2), SEQ ID NO:75 (VH CDR3), SEQ ID NO:78 (VL CDR1), SEQ ID NO:79 (VL CDR2), and SEQ ID NO:80 (VL CDR3).

In some embodiments, the antibody or fragment thereof comprises the amino acid sequences of SEQ ID NO:73 (VH CDR1), SEQ ID NO:74 (VH CDR2), SEQ ID NO:75 (VH CDR3), SEQ ID NO:78 (VL CDR1), SEQ ID NO:79 (VL CDR2), and SEQ ID NO:80 (VL CDR3). In some embodiments, the antibody or fragment thereof comprises the amino acid sequences of SEQ ID NO:72 and SEQ ID NO:77. In some embodiments, the antibody or fragment thereof comprising the amino acid sequences of SEQ ID NO: 72 and SEQ ID NO: 77 is PB2452 (MEDI2452). In some embodiments, the antibody or fragment thereof comprises SEQ ID NO: 81. In some embodiments, the antibody or fragment thereof comprises the amino acid sequences of a VH region and a CH region. In some embodiments, the antibody or fragment thereof comprises the amino acid sequences of a VH region and a CH1 region. In some embodiments, the antibody or fragment thereof comprises the amino acid sequences of SEQ ID NO: 72 and a CH1 region. In some embodiments, the antibody or fragment thereof comprises the amino acid sequence of SEQ ID NO: 83. In some embodiments, the antibody or fragment thereof comprises the amino acid sequence encoded by the nucleic acid sequence of SEQ ID NO: 84. In some embodiments, the antibody or fragment thereof comprises the amino acid sequences of a VL and a CL region. In some embodiments, the antibody or fragment thereof comprises the amino acid sequences of SEQ ID NO: 77 and a CL region. In some embodiments, the antibody or fragment thereof comprises the amino acid sequence of SEQ ID NO: 85. In some embodiments, the antibody or fragment thereof comprises the amino acid sequence encoded by the nucleic acid sequence of SEQ ID NO: 86. In some embodiments, the antibody or fragment thereof comprising the amino acid sequences of SEQ ID NO: 83 and SEQ ID NO: 85 is PB2452 (MEDI2452).

In some embodiments, the antibodies or fragments thereof of the disclosure include one or more amino acid substitutions, deletions or additions, either from natural mutations or human manipulation. As indicated, changes are preferably of a minor nature, such as conservative amino acid substitutions that do not significantly affect the folding or activity of the antibody or fragment thereof.

The present disclosure provides antibodies or fragments thereof that comprise, or alternatively consist of, variants (including derivatives) of the VH domains, VH CDRs, VL domains, and VL CDRs described herein, which antibodies immunospecifically bind to ticagrelor or a derivative or metabolite thereof. Standard techniques known to those of skill in the art can be used to introduce mutations in the nucleotide sequence encoding a molecule of the invention, including, for example, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH domain, VHCDR1, VHCDR2, VHCDR3, VL domain, VLCDR1, VLCDR2, or VLCDR3. In specific embodiments, the variants encode substitutions of VHCDR3. In a preferred embodiment, the variants have conservative amino acid substitutions at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind ticagrelor or a derivative or metabolite thereof). Following mutagenesis, the encoded protein may routinely be expressed and the functional and/or biological activity of the encoded protein, (e.g., ability to immunospecifically bind ticagrelor or a derivative or metabolite thereof) can be determined using techniques described herein or by routinely modifying techniques known in the art.

In another embodiment, an antibody or fragment thereof of the disclosure that immunospecifically binds to ticagrelor, a derivative, or a metabolite thereof comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VL domains. In another embodiment, an antibody of the disclosure that immunospecifically binds to ticagrelor, a derivative, or a metabolite thereof comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VL CDRs. In another embodiment, an antibody of the disclosure that immunospecifically binds to ticagrelor, a derivative, or a metabolite thereof comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VL CDR3s. Nucleic acid molecules encoding these antibodies are also encompassed by the disclosure.

In another embodiment, an antibody or fragment thereof of the disclosure that immunospecifically binds to ticagrelor, a derivative, or a metabolite thereof comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VH domains. In another embodiment, an antibody of the disclosure that immunospecifically binds to ticagrelor, a derivative, or a metabolite thereof comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VH CDRs. In another embodiment, an antibody of the disclosure that immunospecifically binds to ticagrelor, a derivative, or a metabolite thereof comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VH CDR3s. Nucleic acid molecules encoding these antibodies are also encompassed by the disclosure.

PB2452 is a recombinant human IgG1λ monoclonal Fab antibody fragment that binds specifically to ticagrelor and TAM. PB2452 was obtained by optimization of a human anti-ticagrelor antibody using phage display, from libraries that were generated by randomizing amino acids in the variable heavy or variable light chain complementarity-determining region 3s followed by affinity selection and screening. See US 2016/0130366 which is incorporated by reference herein in its entirety for all purposes. PB2452 is produced in E. coli cells and is purified using a 4-step chromatography process.

In some embodiments, the antibody or fragment thereof binds to ticagrelor and neutralizes the anti-platelet aggregation activity of ticagrelor and TAM, thus restoring ADP-induced platelet aggregation in the presence of ticagrelor and TAM.

In some embodiments, the terminal half-life of the antibody or fragment thereof in a subject is about the same as the terminal half-life of ticagrelor and TAM. In some embodiments the antibody terminal half-life is from about 4-24 hours (e.g., 4, 5, 6, 7, 8, 9, 10, 11, 12, 13, 14, 15, 16, 17, 18, 19, 20, 21, 22, 23, or 24 hours). In some embodiments the terminal half-life is from about 4-12 hours (e.g., 4, 5, 6, 7, 8, 9, 10, 11, or 12 hours). In some embodiments, the terminal half-life in a subject is between about 6-9 hours. In some embodiments, the terminal half-life in a subject is between about 6-7 hours. In some embodiments, the terminal half-life is about 6.9 hours.

In some embodiments, the distribution half-life of the antibody or fragment thereof in a subject is about the same as the distribution half-life of ticagrelor and TAM. In some embodiments the distribution half-life is from about 0.1 to 2 hours (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2 hours). In some embodiments the distribution half-life is from about 0.1 to 1 hour (e.g., 0.1, 0.2, 0.3, 0.4, 0.5, 0.6, 0.7, 0.8, 0.9, or 1.0 hour). In some embodiments, the distribution half-life is about 0.89 hours.

In some embodiments, the antibody or fragment thereof provides for a rapid onset of activity. For example, in embodiments the antibody time to onset or the time to neutralize ticagrelor and TAM mediated platelet inhibition, is from about 5-120 minutes, or from about 5-60 minutes. In some embodiments, the time to onset is less than 60 minutes. In some embodiments, the time to onset is about 30 minutes. In some embodiments, the time to onset is less than about 30 minutes. In some embodiments, the time to onset is less than about 10 minutes. In some embodiments, the time to onset is less than about 5 minutes.

In some embodiments, the antibody or fragment thereof provides for sustained inhibition of ticagrelor and TAM activity. In some embodiments, the inhibition of ticagrelor and TAM activity by the antibody or fragment thereof is sustained for about 2 to about 48 hours (e.g. 2, 3, 4, 5, 6, 7, 8, 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours). In some embodiments, the sustained inhibition of ticagrelor and TAM activity is dose dependent. In some embodiments, the sustained inhibition of ticagrelor and TAM activity is dependent on the dose administered in a bolus before IV infusion. In some embodiments, the sustained inhibition of ticagrelor and TAM activity is dependent on the dose administered via IV infusion.

In some embodiments, the antibody or fragment thereof of the present disclosure exhibits both rapid (e.g. within 5 minutes of administration) and sustained (e.g. up to 24 or 48 hours) inhibition of ticagrelor and TAM activity.

In some embodiments, the antibody or fragment thereof is administered to the subject in need thereof immediately after the last ticagrelor administration. In some embodiments, the antibody or fragment thereof is administered to the subject in need thereof within about 1 hour to 120 hours after the last ticagrelor administration. In some embodiments, the antibody or fragment thereof is administered to a subject in need thereof within about 1 hour to 72 hours after the last ticagrelor administration. In some embodiments, the antibody or fragment thereof is administered to the subject in need thereof within about 1 hour to 24 hours (e.g. about 1, 2, 3, 4, 5, 6, 7, 8, 9, 10, 11, 12, 13, 14, 15, 16, 17, 18, 19, 20, 21, 22, 23, or 24 hours).

In some embodiments the antibody or fragment thereof has a PK/PD profile that provides for a rapid offset of activity, such that, for example, a subject who has been administered the antibody may recommence with the prescribed ticagrelor therapy. In some embodiments, a subject who has received an antibody disclosed herein (e.g., by i.v. infusion) may receive or restart ticagrelor therapy within six hours following the administration of the antibody. In some embodiments, a subject who has received an antibody or fragment thereof disclosed herein (e.g., by i.v. infusion) may receive or restart ticagrelor therapy within twelve hours following the administration of the antibody. In some embodiments, a subject who has received an antibody disclosed herein (e.g., by i.v. infusion) may receive or restart ticagrelor therapy within twenty-four hours following the administration of the antibody.

### PB2452 Activity

Without being bound by theory, PB2452 binds to ticagrelor with an affinity that is 100 times stronger than ticagrelor's affinity for the P2Y₁₂ receptor. This high affinity enables PB2452 to bind to free ticagrelor, resulting in a rapid reversal of ticagrelor's effect and restoration of platelet activity.

The chemical starting point for the development of ticagrelor was adenosine triphosphate (ATP), and ticagrelor retains an adenosine-like core. To confirm the specificity of PB2452 for ticagrelor and ticagrelor active metabolite (TAM), its binding to ATP, ADP, and adenosine was evaluated. To further confirm PB2452 specificity, a structural database for marketed drugs was interrogated for molecules that have any structural similarity to ticagrelor. From this in silico analysis, a panel of 12 compounds was selected (fenofibrate, nilvadipine, cilostazol, bucladesine, regadenoson, cyclothiazide, cyfluthrin, lovastatin, linezolid, simvastatin, cangrelor, and pantoprazole). The selectivity of PB2452 was determined by competition binding with PB2452 to biotinylated ticagrelor. No inhibition of PB2452 binding to biotinylated ticagrelor was found for ATP, ADP, adenosine, or the 12 structurally related compounds. Thus, PB2452 binds with high affinity and selectivity to ticagrelor and TAM.

In some embodiments, the anti-ticagrelor antibody or fragment thereof reverses, prevents, inhibits, or reduces ticagrelor or TAM activity. In some embodiments, this ticagrelor or TAM activity is selected from, but not limited to, decreasing ADP-induced platelet aggregation and/or binding to the P2Y₁₂ receptor. In some embodiments, administration of the anti-ticagrelor antibody or fragment thereof restores ADP-induced platelet aggregation and/or binding to the P2Y₁₂ receptor. In some embodiments, the anti-ticagrelor antibody that restores ADP-induced platelet aggregation and/or binding to the P2Y₁₂ receptor is PB2452. See US 2016/0130366 which is incorporated by reference herein in its entirety for all purposes.

### Methods of Treatment

In some aspects, the present disclosure provides a method of reversing, inhibiting, decreasing, or preventing ticagrelor or TAM activity comprising administering pharmaceutical compositions comprising an anti-ticagrelor antibody or fragment thereof to a subject in need. The reversal, inhibition, decrease, or prevention of ticagrelor or TAM activity may be measured by any means known in the art, including, for example by measuring free or total ticagrelor in blood samples

In some embodiments, administration of the pharmaceutical compositions comprising an anti-ticagrelor antibody or fragment thereof restores platelet aggregation. In some embodiments, administration of the pharmaceutical compositions comprising an anti-ticagrelor antibody or fragment thereof inhibits the binding of ticagrelor or TAM to the P2Y₁₂ receptor. In some embodiments, the anti-ticagrelor antibody or fragment thereof is PB2452.

In some aspects, the present disclosure provides a method of restoring platelet aggregation comprising administering pharmaceutical compositions comprising an anti-ticagrelor antibody or fragment thereof to a subject in need. In some aspects, the present disclosure provides a method of decreasing blood loss in a patient receiving ticagrelor comprising administering pharmaceutical compositions comprising an anti-ticagrelor antibody or fragment thereof to a subject in need. In some embodiments, administration of the pharmaceutical composition of the present disclosure inhibits ticagrelor-associated bleeding in a patient.

In some embodiments, administration of pharmaceutical compositions comprising an anti-ticagrelor antibody or fragment thereof to a subject in need clears ticagrelor and/or TAM from the patient's body. In some embodiments, administration of pharmaceutical compositions comprising an anti-ticagrelor antibody or fragment thereof to a subject in need reduces the amount of ticagrelor and/or TAM in a patient's blood. In some embodiments, administration of pharmaceutical compositions comprising an anti-ticagrelor antibody or fragment thereof to a subject in need reduces the amount of ticagrelor in a patient's blood by about 100% to about 5% compared to baseline amounts of ticagrelor and/or TAM. In some embodiments, administration of pharmaceutical compositions comprising an anti-ticagrelor antibody or fragment thereof to a subject in need reduces the amount of ticagrelor in a patient's blood serum by about 90%, about 80%, about 70%, about 60%, about 50%, about 40%, about 30%, about 20%, about 10% or about 5% compared to baseline amounts of ticagrelor and/or TAM.

In some embodiments, administration of pharmaceutical compositions comprising an anti-ticagrelor antibody or fragment thereof to a subject reduces the amount of free ticagrelor and/or TAM in the patient's body. In some embodiments, administration of pharmaceutical compositions comprising an anti-ticagrelor antibody or fragment thereof to a subject in need reduces the amount of free ticagrelor and/or TAM in a patient's blood. In some embodiments, administration of pharmaceutical compositions comprising an anti-ticagrelor antibody or fragment thereof to a subject in need reduces the amount of free ticagrelor and/or TAM in a patient's blood by about 100% to about 15% compared to baseline amounts of ticagrelor and/or TAM. In some embodiments, administration of pharmaceutical compositions comprising an anti-ticagrelor antibody or fragment thereof to a subject in need reduces the amount of free ticagrelor and/or TAM in a patient's blood by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 80%, about 70%, about 60%, about 50%, about 40%, about 30%, about 20%, about 10% or about 5% compared to baseline amounts of ticagrelor and/or TAM. In some embodiments, ticagrelor or TAM activity is reversed, inhibited, decreased, or prevented for about 1 hour to about 2 days. In some embodiments, ticagrelor or TAM activity is reversed, inhibited, decreased, or prevented for about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours or about 48 hours. In some embodiments, this reversal, inhibition, decrease, or prevention is observed at the time points disclosed herein.

In some embodiments, administration of the pharmaceutical compositions disclosed herein reverses, inhibits, decreases, or prevents ticagrelor or TAM activity in a subject compared to an untreated subject. In some embodiments, ticagrelor or TAM activity is reversed, inhibited, decreased, or prevented by about 1%, about 5%, about 10%, about 20%, about 30%, about 40%, about 50%, about 60%, about 70%, about 80%, or about 90% or about 100% compared with activity in an untreated subject. In some embodiments, this reversal, inhibition, decrease, or prevention is observed at the time points disclosed herein.

In some embodiments, administration of the pharmaceutical compositions disclosed herein reverses inhibition of platelet aggregation in a subject compared to an untreated subject on ticagrelor. In some embodiments, the inhibition of platelet aggregation is reversed by about 1%, about 5%, about 10%, about 20%, about 30%, about 40%, about 50%, about 60%, about 70%, about 80%, or about 90% or about 100% compared with inhibition of platelet aggregation in an untreated subject on ticagrelor. In some embodiments, this reversal of inhibition of platelet aggregation is observed at the time points disclosed herein.

In some embodiments, administration of pharmaceutical compositions comprising an anti-ticagrelor antibody or fragment thereof to a subject restores platelet aggregation in the patient's blood. In some embodiments, administration of pharmaceutical compositions comprising an anti-ticagrelor antibody or fragment thereof to a subject in need restores platelet aggregation in a patient's blood to about 100% to about 15% compared to baseline levels of normal platelet aggregation. In some embodiments, administration of pharmaceutical compositions comprising an anti-ticagrelor antibody or fragment thereof to a subject in need restores platelet aggregation in a patient's blood to about 100%, about 90%, about 80%, about 70%, about 60%, about 50%, about 40%, about 30%, about 20%, about 10% or about 5% of baseline levels of normal platelet aggregation. In some embodiments, administration of pharmaceutical compositions comprising an anti-ticagrelor antibody or fragment thereof to a subject in need restores platelet aggregation in a patient's blood to about 80% or greater of baseline levels of normal platelet aggregation. In some embodiments, platelet aggregation is restored to at least 80% of baseline levels of normal platelet aggregation in about 1 hour to about 2 days after administration. In some embodiments, platelet aggregation is restored at about 5 minutes, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours or about 48 hours. In some embodiments, this restoration is observed at the time points disclosed herein.

In some embodiments, administration of pharmaceutical compositions comprising an anti-ticagrelor antibody or fragment thereof to a subject restores platelet aggregation in the patient's blood as measured by the VerifyNow™ P2Y₁₂ (also known as the VerifyNow™ PRUTest) assay method (Accriva/Instrumentation Laboratory, San Diego Calif.). In some embodiments, administration of pharmaceutical compositions comprising an anti-ticagrelor antibody or fragment thereof to a subject in need restores platelet aggregation in a patient's blood as measured by the VerifyNow™ assay to about 50 to about 250 platelet reactivity units (PRU). In some embodiments, administration of pharmaceutical compositions comprising an anti-ticagrelor antibody or fragment thereof to a subject in need restores platelet aggregation in a patient's blood as measured by the VerifyNow™ assay to about 250 platelet reactivity units (PRU), to about 240 PRU, to about 230 PRU, to about 220 PRU, to about 210 PRU to about 200 PRU, to about 190 PRU, to about 180 PRU, to about 170 PRU, to about 160 PRU, to about 150 PRU, to about 140 PUR, to about 130 PRU, to about 120 PRU, to about 110 PRU, to about 100 PRU, to about 90 PRU, to about 80 PRU, to about 70 PRU, to about 60 PRU, to about 50 PRU. In some embodiments, administration of pharmaceutical compositions comprising an anti-ticagrelor antibody or fragment thereof to a subject in need restores platelet aggregation in a patient's blood to at least 180 PRU. In some embodiments, platelet aggregation is restored to at least 180 PRU in about 1 hour to about 2 days after administration. In some embodiments, platelet aggregation is restored to at least 180 PRU at about 5 minutes, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours or about 48 hours. In some embodiments, this restoration is observed at the time points disclosed herein.

In some embodiments, administration of pharmaceutical compositions comprising an anti-ticagrelor antibody or fragment thereof to a subject restores platelet aggregation in the patient's blood as measured by the light transmittance aggregometry (LTA) assay method. In some embodiments, administration of pharmaceutical compositions comprising an anti-ticagrelor antibody or fragment thereof to a subject in need restores platelet aggregation in a patient's blood as measured by the LTA assay to about 50% to about 150% baseline platelet aggregation. In some embodiments, administration of pharmaceutical compositions comprising an anti-ticagrelor antibody or fragment thereof to a subject in need restores platelet aggregation in a patient's blood as measured by the LTA assay to about to 150% baseline platelet aggregation, to about 140% baseline PRI, to about 130% baseline platelet aggregation, to about 120% baseline platelet aggregation, to about 110% baseline platelet aggregation, to about 100% baseline platelet aggregation, to about 90% baseline platelet aggregation, to about 80% baseline platelet aggregation, to about 70% baseline platelet aggregation, to about 60% baseline platelet aggregation, to about 50% baseline platelet aggregation. In some embodiments, administration of pharmaceutical compositions comprising an anti-ticagrelor antibody or fragment thereof to a subject in need restores platelet aggregation in a patient's blood to at least 100% platelet aggregation. In some embodiments, platelet aggregation is restored to at least 100% platelet aggregation in about 1 hour to about 2 days after administration. In some embodiments, platelet aggregation is restored to at least 100% platelet aggregation at about 5 minutes, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours or about 48 hours. In some embodiments, this restoration is observed at the time points disclosed herein.

In some embodiments, administration of pharmaceutical compositions comprising an anti-ticagrelor antibody or fragment thereof to a subject restores platelet aggregation in the patient's blood or P2Y₁₂ receptor signaling as measured by the vasodilator-stimulated phosphoprotein (VASP) method. In some embodiments, administration of pharmaceutical compositions comprising an anti-ticagrelor antibody or fragment thereof to a subject in need restores platelet aggregation in a patient's blood or P2Y₁₂ receptor signaling as measured by VASP to about 50% to about 150% baseline platelet reactivity index (PRI). In some embodiments, administration of pharmaceutical compositions comprising an anti-ticagrelor antibody or fragment thereof to a subject in need restores platelet aggregation in a patient's blood or P2Y₁₂ receptor signaling as measured by VASP to about 150% baseline platelet reactivity index (PRI), to about 140% baseline PRI, to about 130% baseline PRI, to about 120% baseline PRI, to about 110% baseline PRI, to about 100% baseline PRI, to about 90% baseline PRI, to about 80% baseline PRI, to about 70% baseline PRI, to about 60% baseline PRI, to about 50% baseline PRI. In some embodiments, administration of pharmaceutical compositions comprising an anti-ticagrelor antibody or fragment thereof to a subject in need restores platelet aggregation in a patient's blood or P2Y₁₂ receptor signaling to at least 100% baseline PRI. In some embodiments, platelet aggregation or P2Y₁₂ receptor signaling is restored to at least 100% baseline PRI in about 1 hour to about 2 days after administration. In some embodiments, platelet aggregation or P2Y₁₂ receptor signaling is restored to at least 100% baseline PRI at about 5 minutes, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours or about 48 hours. In some embodiments, this restoration is observed at the time points disclosed herein.

### Patient Populations

The anti-ticagrelor antibodies or fragments thereof of the present disclosure may be administered to any patient in need. In some embodiments, the patient is at risk of, or has been diagnosed with, Acute Coronary Syndrome (ACS). In some embodiments, the patient is at risk of, or has been diagnosed with myocardial infarction (MI). In some embodiments, the patient has a history of MI. In some embodiments, the patient is receiving, or has received ticagrelor. In some embodiments, the patient is receiving, or has received ticagrelor along with another anti-platelet therapy, such as aspirin.

In some embodiments, the patient has unstable angina, stable ischemic heart disease, in sickle cell disease, including pediatric patients, atrial fibrillation, coronary arterial disease, peripheral arterial disease, ischemic stroke, one or more coronary stents, carotid artery stents, stents following an intracranial aneurysm, or arterio-venous fistulae created for hemodialysis.

In some embodiments, the patient has type 2 diabetes mellitus. In some embodiments, the patient has type 2 diabetes mellitus and coronary disease. In some embodiments, the patient has type 2 diabetes mellitus with a history of percutaneous coronary intervention.

In some embodiments, the patient is at higher risk or increased rate of bleeding associated with ticagrelor treatment. In some embodiments, this ticagrelor-associated bleeding is gastrointestinal bleeding. In some embodiments, this ticagrelor-associated bleeding is intracranial bleeding, or intracranial hemorrhage (ICH). In some embodiments, this ticagrelor-associated bleeding is as a result of traumatic injury, such as a road traffic accident. In some embodiments, the ticagrelor-associated bleeding is categorized as major bleeding. Major bleeding will include any bleeding event which is judged by the treating physician to require reversal. This includes, but is not limited to, bleeding events which are life-threatening, potentially leading to clinically significant disability, requiring surgery to control the bleeding, requiring treatment with blood products, or acute bleeding associated with a clinically important drop in hemoglobin levels.

In some embodiments, the patient requires urgent surgery or intervention. Urgent surgery or intervention is defined as requirement for a surgical operation or medical procedure associated with a risk or perioperative bleeding in a situation where it is not medically advisable to withhold ticagrelor five days. Requirement for urgent surgery may include, but is not limited to, patients in any of the following clinical situations: undergoing surgery or procedures known to be associated with a significant risk of bleeding (such as coronary artery bypass surgery); undergoing surgery or procedures which may have an adverse surgical outcome if bleeding is not carefully controlled (such as neurological, ophthalmologic, or joint replacement surgery); at risk of experiencing perioperative events such as shock, myocardial infarction or stroke if significant perioperative bleeding occurs (especially in elderly patients or those with co-morbidities); at high risk of thrombosis if dual antiplatelet therapy is withheld preoperatively (such as patients with recent coronary stent placement).

In some embodiments, the patient has begun experiencing bleeding before administration of an anti-ticagrelor antibody or fragment thereof. In some embodiments, the patient has not begun experiencing bleeding before administration of an anti-ticagrelor antibody or fragment thereof. In some embodiments, the patient requires surgery, and thus poses increased risk of bleeding due to ticagrelor treatment. In some embodiments, the surgery is urgent surgery. In some embodiments the surgery is emergent surgery.

In some embodiments, the patient is an adult. In some embodiments, the adult patient between 30 and 100 years old or more. In some embodiments, the adult patient is over 40 years old, over 50 years old, over 60 years, over 70 years old, over 80 years old, or over 90 years old. In some embodiments, the adult patient is between 50-64 years old. In some embodiments, the adult patient is between 65-75 years old. In some embodiments, the patient is defined as older (e.g. between the ages of 50 and 64 years old inclusive). In some embodiments, the patient is defined as elderly (e.g. over the age of 65 years or between the ages of 65 and 80 years old inclusive). In some embodiments, the older or elderly patient has been pretreated with ticagrelor and aspirin. In some embodiments, older or elderly patients experience higher exposure to ticagrelor and/or a lower response to ticagrelor compared to younger subjects.

In some embodiments, the patient is a young adult. In some embodiments, the young adult patient between 18 and 30 years old or more. In some embodiments, the patient is a pediatric patient under 18 years of age. In some embodiments, the patient is a pediatric patient under 2 years of age. In some embodiments, the pediatric patient or young adult patient has sickle cell disease.

### Pharmaceutical Compositions and Administration

The present disclosure provides pharmaceutical compositions including an anti-ticagrelor antibody or fragment thereof with one or more pharmaceutically acceptable excipients and/or diluents. In some embodiments, the anti-ticagrelor antibody or fragment thereof is PB2452.

The formulations of the present disclosure may include any appropriate excipient known in the art. Exemplary excipients include, but are not limited to, amino acids such as histidine, glycine, or arginine; glycerol; sugars, such as sucrose; surface active agents such as polysorbate 20 and polysorbate 80; citric acid; sodium citrate; antioxidants; salts including alkaline earth metal salts such as sodium, potassium, and calcium; counter ions such as chloride and phosphate; sugar alcohols (e.g. mannitol); preservatives; sugar alcohols (e.g. mannitol, sorbitol); and buffering agents. Exemplary salts include sodium chloride, potassium chloride, magnesium chloride, calcium chloride, sodium phosphate dibasic, sodium phosphate monobasic, sodium phosphate, and potassium phosphate.

In certain embodiments, the formulation may include from about 5 mM histidine/histidine hydrochloride buffer to about 100 mM histidine/histidine hydrochloride buffer. In some embodiments, the formulation includes about 50 mM histidine/histidine hydrochloride buffer, about 40 mM histidine/histidine hydrochloride buffer, about 30 mM histidine/histidine hydrochloride buffer, about 25 mM histidine/histidine hydrochloride buffer, about 20 mM histidine/histidine hydrochloride buffer, or about 15 mM histidine/histidine hydrochloride buffer.

In certain embodiments, the formulation may include from about 100 mM sucrose to about 1 M sucrose. In some embodiments, the formulation may include about 150 mM sucrose, about, about 200 mM sucrose, about 250 mM sucrose, about 290 mM sucrose, about 300 mM sucrose, about 350 mM sucrose, about 400 mM sucrose, or about 500 mM sucrose.

In certain embodiments, the formulation may include a surfactant. In some embodiments the surfactant is a non-ionic surfactant. In some embodiments, the non-ionic surfactant is polysorbate 80. In some embodiments, the formulation may include from about 0.01% w/v polysorbate 80 to about 1.00% w/v polysorbate 80. In some embodiments, the formulation may include about 0.01% w/v, about 0.02% w/v, about 0.03% w/v, about 0.05% w/v, about 0.06% w/v, about 0.07% w/v, about 0.08% w/v, about 0.09% w/v, or about 0.1% w/v polysorbate 80.

In certain embodiments, the formulation may include from about 10 mg/mL anti-ticagrelor antibody or fragment thereof to about 200 mg/mL anti-ticagrelor antibody or fragment thereof. In some embodiments, the formulation may include about 10 mg/mL, about 20 mg/mL, about 30 mg/mL, about 40 mg/mL, about 50 mg/mL, about 60 mg/mL, about 70 mg/mL, about 80 mg/mL, about 90 mg/ml, about 100 mg/mL, about 110 mg/mL, about 120 mg/mL, about 130 mg/mL, about 140 mg/mL, or about 150 mg/mL anti-ticagrelor antibody or fragment thereof.

In some embodiments, the formulation includes 100 mg/mL of the anti-ticagrelor antibody or fragment thereof, 25 mM histidine/histidine hydrochloride buffer, 290 mM sucrose, and 0.05% (w/v) polysorbate 80, pH 6.0.

The formulation can be stored frozen, refrigerated or at room temperature. The storage condition may be below freezing, such as lower than about −10° C., or lower than about −20° C., or lower than about −40° C., or lower than about −70° C. Storage conditions are generally less than the room temperature, such as less than about 32° C., or less than about 30° C., or less than about 27° C., or less than about 25° C., or less than about 20° C., or less than about 15° C. In some embodiments, the formulation is stored at 2°−8° C. For example, the formulation may be isotonic with blood or have an ionic strength that mimics physiological conditions

In some embodiments, the formulation is formulated at physiological pH. In some embodiments, the formulation is formulated at a pH in the range of about 5.5 to about 8.5. In some embodiments, the formulation is formulated at a pH in the range of about 6.0 to about 8.0. In some embodiments, the formulation is formulated at a pH in the range of about 6.5 to about 7.5. In some embodiments, the formulation is formulated at a pH of 7.5. In some embodiments, formulations with a lower pH demonstrate improved formulation stability compared to formulations at a higher pH. In some embodiments, formulations with a higher pH demonstrate improved formulation stability compared to formulations at a lower pH.

In some embodiments, the formulation is stable at storage conditions. Stability can be measured using any appropriate means in the art. Generally, a stable formulation is one that shows less than a 5% increase in degradation products or impurities. In some embodiments, the formulation is stable for at least about 1 month, at least about 2 months, at least about 3 months, at least about 4 months, at least about 5 months, at least about 6 months, at least about one year, or at least about 2 years or more at the storage conditions. In some embodiments, the formulation is stable for at least about 1 month, at least about 2 months, at least about 3 months, at least about 4 months, at least about 5 months, at least about 6 months, or at least about one year or more at 25° C.

In some aspects, the formulation is a lyophilized product. In some embodiments, the formulation is a lyophilized product containing about 1 g to about 36 g of the anti-ticagrelor antibody or fragment thereof. In some embodiments, the formulation is a lyophilized product containing about 6 g of the anti-ticagrelor antibody or fragment thereof. In some aspects, following reconstitution with water for injection, the product may be further diluted into 0.9% saline for intravenous (iv) infusion. In some embodiments, the product is not lyophilized and is further diluted into 0.9% saline for intravenous (iv) infusion.

In one aspect, the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). In certain specific aspects, the endotoxin and pyrogen levels in the composition are less than about 1 EU/mg, or less than about 0.1 EU/mg, or less than about 0.01 EU/mg, or less than about 0.001 EU/mg. In some embodiments, the endotoxin and pyrogen levels in the composition are 0.0138 EU/mg or less.

When used for in vivo administration, the formulations of the disclosure should be sterile. The formulations of the disclosure may be sterilized by various sterilization methods, including sterile filtration, radiation, etc. In one aspect, the formulation is filter-sterilized with a pre-sterilized 0.22-micron filter. Sterile compositions for injection can be formulated according to conventional pharmaceutical practice as described in “Remington: The Science & Practice of Pharmacy”, 21^{st} ed., Lippincott Williams & Wilkins, (2005).

In some embodiments, the pharmaceutical composition is formulated for intravenous administration. In some embodiments, the formulation is administered intravenously over about 5 minutes to 48 hours. In some embodiments, the formulation is administered in any appropriate volume. In some embodiments, the formulation is administered in a total volume of about 30 mL to about 2 L. In some embodiments, the formulation is administered in a total volume of about 30 mL, about 40 mL, about 50 mL, about 100 mL, about 125 mL, about 150 mL, about 175 mL, about 200 mL, about 225 mL, about 250 mL, about 275 mL, about 300 mL, about 400 mL, about 500 mL, about 1 L, about 1.5 L, or about 2 L. In some embodiments, the formulation is administered intravenously over about 30 minutes in a total volume of about 250 mL. In some embodiments, the formulation is first administered as a bolus, followed by a longer infusion. In some embodiments, the longer infusion following the bolus is about 4 hours. In some embodiments, the longer infusion following the bolus is about 8 hours. In some embodiments, the longer infusion following the bolus is about 12 hours. In some embodiments, the longer infusion following the bolus is about 18 hours. In some embodiments, the longer infusion following the bolus is about 24 hours. In some embodiments, the longer infusion following the bolus is about 36 hours.

In some embodiments, the concentration of anti-ticagrelor antibody or fragment thereof in the formulation varies between 0.4 mg/mL up to 72 mg/mL in a single IV infusion, 250 mL to be delivered over 30 minutes to 12 hours in doses of 0.1 g, 0.3 g, 1.0 g, 3 g, 9 g, 180 g, 24 g, 30 g, 36 g or 48 g or intermediate doses between 9 to 48 g. In some cases, a portion of the therapeutic composition is infused (up to about 12 g) at a faster rate (equivalent to a bolus) for the first 5-20 minutes of the infusion.

In some embodiments, the anti-ticagrelor antibody or fragment thereof is stored in one or more glass vials and subsequently transferred to an infusion bag for administration. In some embodiments, the anti-ticagrelor antibody or fragment thereof is stored in one or more glass vials and subsequently transferred to a syringe for administration using a syringe pump. In some embodiments, the anti-ticagrelor antibody or fragment thereof is stored in pre-filled syringe for administration using a syringe pump. In some embodiments, the anti-ticagrelor antibody or fragment thereof is stored in an IV container, such as Baxter Galaxy Liquid Premix System or Baxter Galaxy Frozen Premix System.

In some embodiments, the antibody or fragment thereof is administered to effect rapid and prolonged reversal of ticagrelor activity. In some embodiments, the infusion rate remains constant over the entire infusion. In some embodiments, the infusion rate varies over the infusion time. In some embodiments, a greater amount of the pharmaceutical composition is administered first in the infusion, and the amount is tapered during the rest of the infusion.

In some embodiments, the infusion duration lasts between about 5 minutes and about 36 hours. In some embodiments, the infusion regimen is selected from, but not limited to infusion of about 3 g to about 36 g at a constant infusion rate over about 1 hour to about 24 hours, infusion of about 3 g over about 5 minutes, followed by infusion of about 15 grams over about 8 hours, infusion of about 6 g over about 15 minutes, followed by infusion of about 6 grams over about 3 hours, followed by infusion of about 6 g over about 8.75 hours, infusion of about 6 g over about 15 minutes, followed by infusion of about 6 grams over about 4 hours, followed by infusion of about 6 g over about 12 hours, infusion of about 6 g over about 10 minutes, followed by infusion of about 6 grams over about 3 hours, followed by infusion of about 6 g over about 13 hours, infusion of about 12 g over about 10 minutes, followed by infusion of about 12 grams over about 6 hours, followed by infusion of about 12 g over about 18 hours.

In some embodiments, if rapid reversal of ticagrelor activity is desired (e.g. during an active bleed in a patient), the antibody or fragment thereof of the present disclosure may be administered according to the below:

1. about 3 g to about 6 g infused over about 5 to about 15 minutes, followed by about 3 g to about 6 g infused over about 1 to about 3 hours, followed by about 3 g to about 6 g infused over about 3 to about 8 hours.

2. about 3 g to about 6 g infused over about 5 to about 15 minutes, followed by about 6 g to about 12 g infused over about 1 to about 3 hours, followed by about 6 g to about 12 g infused over about 3 to about 8 hours.

3. about 3 g to about 6 g infused over about 5 to about 15 minutes, followed by about 6 g to about 12 g infused over about 1 to about 3 hours, followed by about 1 g/hour infused over up to about 24 to about 48 hours.

4. about 9 g infused over about 5 to about 30 minutes, followed by about 1 g/hr to about 3 g/hr infused over about 3 to about 8 hours.

5. about 9 g to about 24 g infused over about 1 to about 4 hours.

In some embodiments, if the patient is about to undergo surgery, the antibody or fragment thereof of the present disclosure may be administered according to the below:

1. about 3 to about 6 g infused over about 5 to about 30 minutes, followed by about 3 to about 6 g infused over about 3 to about 6 hours, followed by about 1 g/hr infused over up to about 12 to about 24 hours;

2. about 3 to about 6 g infused over about 5 to about 15 minutes, followed by about 3 to about 6 g infused over about 1 to about 3 hours, followed by about 1 g/hr infused over about 12 to about 24 hours.

3. about 9 g to about 24 g infused over about 1 to about 4 hours.

In some embodiments, if the patient is taking one or more additional drugs that impact ticagrelor exposure, such as drugs that inhibit the activity of cytochrome P450 isoform 3A (CYP3A), leading to increased exposure to ticagrelor, or the patient has taken an overdose of ticagrelor, the antibody or fragment thereof of the present disclosure may be administered according to the below:

1. about 6 to about 12 g infused over about 5 to about 30 minutes, followed by about 6 to about 12 g infused over about 3 to about 6 hours, followed by about 6 to about 12 g infused over up to about 12 to about 24 hours;

2. about 6 to about 12 g infused over about 5 to about 15 minutes, followed by about 6 to about 12 g infused over about 1 to about 3 hours, followed by about 0.5-1 g/hr infused over about 12 to about 24 hours.

3. about 18 g to about 36 g infused over about 3 to about 6 hours.

The formulations may conveniently be presented in unit dosage form and may be prepared by any method known in the art of pharmacy. Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient (e.g., “a therapeutically effective amount”). The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Suitable dosages may range from about 1000 mg to about 36 g, or from about 9 g to about 24 g, or from about 9 g to about 15 g, or from about 1 g to about 3 g. In some embodiments, the dose may be about 1000 mg, about 3 g, about 9 g, about 18 g, about 24 g, about 30 g, about 36 g, or about 48 g. In some embodiments, the amount of anti-ticagrelor antibody or fragment thereof administered to a patient depends on the amount of ticagrelor the patient has received. In some embodiments, the amount of anti-ticagrelor antibody or fragment thereof administered to a patient depends on the body weight of the patient.

The dose of anti-ticagrelor antibody or fragment thereof administered will be that dose which causes the reversal of ticagrelor-induced platelet disaggregation in 95% of the simulated patient population, with reversal taken to be the reversal of the platelet disaggregation to less than 10% of baseline.

In some embodiments, the patient has been administered at least 180 mg ticagrelor. In some embodiments, the patient has been administered a loading dose of at least 180 mg ticagrelor with 90 mg subsequently administered twice a day. In some embodiments, the patient has been administered ticagrelor at least three days prior to administration of an anti-ticagrelor antibody or fragment thereof. In some embodiments, the patient is administered ticagrelor at the same time as administration of an anti-ticagrelor antibody or fragment thereof. In some embodiments, the patient has been administered an overdose of ticagrelor.

Note that the disclosure similarly contemplates that formulations suitable for diagnostic and research use may also be made. The concentration of active agent in such formulations, as well as the presence or absence of excipients and/or pyrogens can be selected based on the particular application and intended use.

It should be understood that singular forms such as “a,” “an,” and “the” are used throughout this application for convenience, however, except where context or an explicit statement indicates otherwise, the singular forms are intended to include the plural. All numerical ranges should be understood to include each and every numerical point within the numerical range, and should be interpreted as reciting each and every numerical point individually. The endpoints of all ranges directed to the same component or property are inclusive, and intended to be independently combinable.

The term “about” when used in connection with a referenced numeric indication means the referenced numeric indication plus or minus up to 10% of that referenced numeric indication. For example, the language “about 50” covers the range of 45 to 55.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein.

### Examples of Non-Limiting Embodiments of the Disclosure

Embodiments of the present subject matter disclosed herein may be beneficial alone or in combination, with one or more other embodiments. Without limiting the foregoing description, certain non-limiting embodiments of the disclosure, numbered 1-57 are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

Embodiment 1. A method of modeling, simulating, and/or determining an effective dosing regimen of an antibody or fragment thereof that binds an inhibitor of P2Y12 receptor signaling or P2Y₁₂ receptor-induced platelet aggregation in a patient population, the method comprising: a) determining a pharmacokinetic-pharmacodynamic (PD/PD) model that characterizes the relationship between ticagrelor and ticagrelor active metabolite (TAM) individually versus P2Y₁₂ receptor-induced platelet aggregation and P2Y₁₂ receptor signaling; b) wherein if the determination in (a) indicates that the P2Y₁₂ receptor-induced platelet aggregation and P2Y₁₂ receptor signaling is decreasing, the predicted effective dosing regimen is a higher dose of the antibody or fragment thereof infused at a faster rate or over a longer period of time; or c) wherein if the determination in (a) indicates that the P2Y₁₂ receptor-induced platelet aggregation and P2Y₁₂ receptor signaling is increasing, the predicted effective dosing regimen is a lower dose of the antibody or fragment thereof and/or infused at a slower rate or over a shorter period of time.

Embodiment 2. The method of embodiment 1, wherein the dosing regimen is sufficient to increase P2Y₁₂ receptor-induced platelet aggregation and P2Y₁₂ receptor signaling values towards the baseline observed before administration of the inhibitor of the P2Y₁₂ receptor signaling.

Embodiment 3. The method of embodiment 1 or embodiment 2, wherein the dosing regimen is effective to sustain the increase of P2Y₁₂ receptor-induced platelet aggregation and P2Y₁₂ receptor signaling.

Embodiment 4. The method of any of embodiments 1-3, wherein P2Y₁₂ receptor-induced platelet aggregation and/or P2Y₁₂ receptor signaling is determined by one or more methods selected from light transmittance aggregometry (LTA), VerifyNow™-based P2Y₁₂ reactivity units (PRU), vasodilatory stimulated phosphoprotein (VASP) phosphorylation, and/or other platelet-function or P2Y₁₂-receptor-signaling assays.

Embodiment 5. The method of any of embodiments 1-4, wherein the metabolism of ticagrelor to TAM is modeled as a function of the concentration values of the antibody or fragment thereof.

Embodiment 6. The method of any of embodiments 1-5, wherein the pharmacokinetic-pharmacodynamic (PK/PD) model that characterizes the relationship between ticagrelor and ticagrelor active metabolite (TAM) individually versus P2Y₁₂ receptor-induced platelet aggregation or LTA and or P2Y₁₂ receptor signaling is determined using the following equation:

$$PRU = Base * ( 1 - \frac{E {max}_{1} * {TICA}^{γ}}{EC {50}_{1}^{γ} + {TICA}^{γ}} - \frac{E {max}_{2} * {TAM}^{γ}}{EC {50}_{2}^{γ} + {TAM}^{γ}} )$$

Embodiment 7. The method of any of embodiments 1-6, wherein the predicted effective dosing regimen comprises an initial bolus followed by a higher rate infusion, and then followed by a slower rate infusion.

Embodiment 8. The method of any of embodiments 1-7, wherein the values of P2Y₁₂ receptor-induced platelet aggregation and P2Y₁₂ receptor signaling necessary for the intended patient population are maintained.

Embodiment 9. The method of any of embodiments 1-8, wherein the P2Y₁₂ receptor-induced platelet aggregation and P2Y₁₂ receptor signaling levels are maintained for about 1 to 48 hours.

Embodiment 10. The method of any of embodiments 1-9, wherein the P2Y₁₂ receptor-induced platelet aggregation and P2Y₁₂ receptor signaling levels are maintained for about 10-30 hours.

Embodiment 11. The method of any of embodiments 1-10, wherein the P2Y₁₂ receptor-induced platelet aggregation and P2Y₁₂ receptor signaling levels are maintained for about 20-24 hours.

Embodiment 12. The method of any of embodiments 1-11, wherein the dosing regimen provides complete reversal of the inhibitor of a P2Y₁₂ receptor-induced platelet aggregation and P2Y₁₂ receptor signaling.

Embodiment 13. The method of any of embodiments 1-12, wherein the dosing regimen provides complete reversal of the inhibitor of a P2Y₁₂ receptor-induced platelet aggregation and P2Y₁₂ receptor signaling within about 5 minutes of infusion onset.

Embodiment 14. The method of any of embodiments 1-13, wherein the complete reversal is sustained for at least 20 to 24 hours.

Embodiment 15. The method of any of embodiments 1-14, wherein administration of the antibody or fragment thereof restores platelet function.

Embodiment 16. The method of any of embodiments 1-15, wherein administration of the antibody or fragment thereof restores platelet aggregation or platelet receptor signaling.

Embodiment 17. The method of any of embodiments 1-16, wherein administration of the antibody or fragment thereof restores platelet aggregation or platelet receptor signaling to at least 80% of baseline.

Embodiment 18. The method of any of embodiments 1-17, wherein administration of the antibody or fragment thereof restores platelet aggregation or platelet receptor signaling within 1 minute to 60 minutes of administration.

Embodiment 19. The method of any of embodiments 1-18, wherein administration of the antibody or fragment thereof restores platelet aggregation or platelet receptor signaling within 5 minutes of administration.

Embodiment 20. The method of any of embodiments 1-19, wherein administration of the antibody or fragment thereof provides a sustained restoration of platelet aggregation or platelet receptor signaling.

Embodiment 21. The method of any of embodiments 1-20, wherein the restoration of platelet aggregation or platelet receptor signaling is sustained for at least 12 hours after administration.

Embodiment 22. The method of any of embodiments 1-21, wherein the restoration of platelet aggregation or platelet receptor signaling is sustained for at least 16 hours after administration.

Embodiment 23. The method of any of embodiments 1-22, wherein the restoration of platelet aggregation or platelet receptor signaling is sustained for at least 24 hours after administration.

Embodiment 24. The method of any of embodiments 1-23, wherein the antibody or fragment thereof is a Fab and the patient is administered a dose between about 1 g and about 48 g.

Embodiment 25. The method of any of embodiments 1-24, wherein the dose is between about 9 g to about 18 g of the Fab.

Embodiment 26. The method of any of embodiments 1-25, wherein the patient is administered a dose of about 1 g, about 3 g, about 9 g, about 18 g, about 24 g, about 30 g, about 36 g or about 48 g of the Fab.

Embodiment 27. The method of any of embodiments 1-26, wherein the antibody or fragment thereof is administered to the patient intravenously.

Embodiment 28. The method of any of embodiments 1-27, wherein the antibody or fragment thereof is administered intravenously over about 15 minutes to about 36 hours.

Embodiment 29. The method of any of embodiments 1-28, wherein the pharmaceutical composition is administered in two or more segments.

Embodiment 30. The method of any of embodiments 1-29, wherein the first segment is a bolus.

Embodiment 31. The method of any of embodiments 1-30, wherein the administration rates for each of the segments differ.

Embodiment 32. The method of any of embodiments 1-31, wherein the administration rates for each of the segments differ for successive segments of the infusion.

Embodiment 33. The method of any of embodiments 1-32, wherein the antibody or fragment thereof is administered in three or more segments, wherein the administration rates for each of the segments differ for successive segments of the infusion.

Embodiment 34. The method of any of embodiments 1-33, wherein the predicted effective dosing regimen of the antibody or fragment thereof comprises a 6 gram IV bolus followed by 6 grams infused over 4 hours, and then 6 grams over 12 hours.

Embodiment 35. The method of any of embodiments 1-34, wherein the antibody or fragment thereof is in a pharmaceutical composition comprising about 50 mg/mL to about 200 mg/mL of the anti-ticagrelor antibody or fragment thereof, about 5 mM to about 50 mM histidine/histidine hydrochloride buffer, about 100 mM to about 300 mM sucrose, and about 0.01% (w/v) to about 1.0% (w/v) polysorbate 80, pH 5.5 to 6.5

Embodiment 36. The method of any of embodiments 1-36, wherein the pharmaceutical formulation comprises 100 mg/mL of the anti-ticagrelor antibody or fragment thereof, 25 mM histidine/histidine hydrochloride buffer, 290 mM sucrose, and 0.05% (w/v) polysorbate 80, pH 6.0.

Embodiment 37. The method of any of embodiments 1-36, wherein the pharmaceutical formulation is diluted in saline for administration.

Embodiment 38. The method of any of embodiments 1-37, wherein the inhibitor of a P2Y12 receptor signaling is ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-ydroxyethoxy)cyclopentane-1,2-diol) or a metabolite or derivative thereof.

Embodiment 39. The method of any of embodiments 1-38, wherein the antibody or a fragment thereof comprises complementarity-determining region (CDR) combinations selected from the group consisting of:
a) SEQ ID NO:53 (VH CDR1), SEQ ID NO:54 (VH CDR2), SEQ ID NO:55 (VH CDR3), SEQ ID NO:58 (VL CDR1), SEQ ID NO:59 (VL CDR2), and SEQ ID NO:60 (VL CDR3);
b) SEQ ID NO:63 (VH CDR1), SEQ ID NO:64 (VH CDR2), SEQ ID NO:65 (VH CDR3), SEQ ID NO:68 (VL CDR1), SEQ ID NO:69 (VL CDR2), and SEQ ID NO:70 (VL CDR3); and
c) SEQ ID NO:73 (VH CDR1), SEQ ID NO:74 (VH CDR2), SEQ ID NO:75 (VH CDR3), SEQ ID NO:78 (VL CDR1), SEQ ID NO:79 (VL CDR2), and SEQ ID NO:80 (VL CDR3).

Embodiment 40. The method of any of embodiments 1-39, wherein the antibody or a fragment thereof comprises a combination of heavy chain variable region (VH) and light chain variable region (VL) sequences selected from the group consisting of SEQ ID NO:52 and SEQ ID NO:57; SEQ ID NO:62 and SEQ ID NO:67; and SEQ ID NO:72 and SEQ ID NO:77.

Embodiment 41. A method of treating a patient using the predicted effective dosing regimen of the method of any of embodiments 1-40.

Embodiment 42. The method of any of embodiments 1-41, wherein the patient has been administered ticagrelor ((1S,2S,3R,5S)-3-[7-{[(1R,2S)-2-(3,4-difluorophenyl)cyclopropyl]amino}-5-(propylthio)-3H-[1,2,3]triazolo[4,5-d]pyrimidin-3-yl]-5-(2-ydroxyethoxy)cyclopentane-1,2-diol).

Embodiment 43. The method of any of embodiments 1-42, wherein the patient is experiencing or at risk of experiencing ticagrelor-associated bleeding.

Embodiment 44. The method of any of embodiments 1-43, wherein the bleeding is major bleeding.

Embodiment 45. The method of any of embodiments 1-44, wherein the bleeding is characterized by being major bleeding or life-threatening bleeding, potentially leading to clinically significant disability, requiring surgery to control the bleeding, requiring treatment with blood products, or is acute bleeding associated with a clinically important drop in hemoglobin.

Embodiment 46. The method of any of embodiments 1-45, wherein the patient requires urgent surgery or intervention.

Embodiment 47. The method of any of embodiments 1-46, wherein the urgent surgery or intervention is known to be associated with a significant risk of bleeding, such as coronary artery bypass surgery, has an adverse surgical outcome if bleeding is not carefully controlled, such as neurological, ophthalmologic, or joint replacement surgery, is associated with risk of experiencing perioperative events; or is indicated in a patient at high risk of thrombosis if dual antiplatelet therapy is withheld perioperatively.

Embodiment 48. The method of any of embodiments 1-47, wherein the patient is at risk of developing, or has been diagnosed with Acute Coronary Syndrome (ACS).

Embodiment 49. The method of any of embodiments 1-48, wherein the patient is at risk of developing, or has been diagnosed with a disease selected from the group consisting of myocardial infarction (MI), unstable angina, stable ischemic heart disease, in sickle cell disease, including pediatric patients, atrial fibrillation, coronary arterial disease, peripheral arterial disease, ischemic stroke, one or more coronary stents, carotid artery stents, stents following an intracranial aneurysm, and arterio-venous fistulae created for hemodialysis.

Embodiment 50. The method of any of embodiments 1-49, wherein the patient is a pediatric patient.

Embodiment 51. The method of embodiment 50, wherein the pediatric patient is younger than 18 years old.

Embodiment 52. The method of embodiment 51, wherein the pediatric patient is younger than 2 years old.

Embodiment 53. The method of any of embodiments 1-49, wherein the patient is an adult patient.

Embodiment 54. The method of embodiment 53, wherein the adult patient is between 18 and 64 years old inclusive.

Embodiment 55. The method of embodiment 54, wherein the patient is over 65 years old.

Embodiment 56. The method of embodiment 55, wherein the patient is between 65 and 80 years old inclusive.

Embodiment 57. The method of any of embodiments 1-56, wherein the patient has been administered aspirin (acetylsalicylic acid).

This disclosure is further illustrated by the following non-limiting examples.

### EXAMPLES

Example 1—Population Pharmacokinetic-Pharmacodynamic Modeling of PB2452, a Monoclonal Antibody Fragment that Reverses Ticagrelor

PB2452 (formerly MEDI2452) is a neutralizing monoclonal antibody fragment that binds ticagrelor (TICA) and its major active circulating metabolite (TAM) with high affinity (Buchanan, Newton, Pehrsson, & et al., 2015). A first-in-human clinical trial was conducted to determine if PB2452 could be used to reverse rapidly the antiplatelet effects of ticagrelor, thereby reducing the risk or severity of bleeding (Bhatt, et al., 2019).

The goal of the present analysis is to fit a population pharmacokinetic-pharmacodynamic (PK/PD) model to characterize the relationship between PB2452, TICA (ticagrelor), TAM, and change in platelet aggregation and P2Y₁₂ receptor signaling, as measured by light transmittance aggregometry (LTA) which assesses inhibition of platelet aggregation (IPA), the VerifyNow™ P2Y₁₂ assay which assesses P2Y₁₂ reactivity units (PRU), and the enzyme-linked immunosorbent assay (ELISA)-based vasodilator stimulated phosphoprotein (VASP) phosphorylation assay which assesses receptor signaling with the platelet reactivity index (PRI). This model was developed during the conduct of the first-in-human study and used to inform dosing decisions throughout the study. The model was then updated with final data from the trial and will be used to inform subsequent early-phase clinical trials being conducted.

### Material and Methods

Study Design:

A single-center, randomized, double-blind, placebo-controlled, single-ascending-dose, phase 1 trial was conducted to evaluate the safety, efficacy, and pharmacokinetic profiles of PB2452 in healthy volunteers 18 to 50 years of age who were pretreated with ticagrelor. The study had 10 cohorts of healthy volunteers. Table 1 shows the dosing regimen for each cohort.

Subjects in Cohorts 1 through 3 did not receive ticagrelor. Subjects in Cohorts 4-10 were pretreated with ticagrelor for 48 hours. The first dose of ticagrelor was an oral loading dose of 180 mg followed by 90 mg administered orally twice daily for 2 days. Subjects in Cohorts 4 through 6 were administered the study drug (PB2452) intravenously immediately after the 5th ticagrelor dose, whereas subjects in Cohorts 7 through 10 were administered study 2 hours after the 5th ticagrelor dose at the time of peak ticagrelor concentration.

Further details on the design and conduct of the study are provided in Bhatt, et al., 2019.

Data Assembly:

All data used in the PK/PD analysis were obtained from subjects in the clinical trial who received ticagrelor and/or PB2452. Subjects who received ticagrelor alone (ticagrelor with placebo) were included in the development of the model.

Ticagrelor (TICA) concentrations, PB2452 concentrations, ticagrelor active metabolite (TAM) concentrations, demographic information, and measures of platelet aggregation (PRU, LTA, VASP) were used to build NONMEM (version 7.4, ICON Development Solutions) input data for the PK/PD analysis. The data consist of Total Ticagrelor (including the TICA-PB2452 complex, protein-bound TICA and free TICA), Total TAM (including the TAM-PB2452 complex, protein-bound TAM and free TAM), uncomplexed PB2452, and Total PB2452 (including uncomplexed PB2452, the TICA-PB2452 complex and the TAM-PB2452 complex) along with the PD measures of platelet aggregation and activation (PRU/LTA/VASP).

Prior to modeling, PB2452, TICA, and TAM concentrations were converted to nanomolar (nM) units using the molecular weights for the analytes.

Time is defined as the time following the first administration of TICA (except for cohorts 1-3, where it is time after first administration of PB2452 since TICA was not given). Depending on the cohort, PB2452 is administered at either 48 hours (cohorts 4-6) or 50 hours (cohorts 7-10).

Data Analysis:

Population PK and PK/PD analyses were carried out using NONMEM version 7.4, PDx-Pop version 5.2 and Intel Visual Fortran Compiler version 12 on Microsoft Windows 10 Professional.

The models described in the following sections are non-linear hierarchical models that were fit using Bayesian Markov Chain Monte-Carlo (MCMC) techniques. If θ and φj represent vectors of individual PK and PD parameters, respectively, then it was assumed that they follow distributions with population parameters Θ and Φ, respectively. The parameters Θ and Φ were then assigned prior distributions according to the prior information available. The Bayesian analysis involved the estimation of the joint distribution of all parameters conditional on the observed data: p(θ, φ, Θ, Φ|PK and PD data), where θ and φ denote collections of all individual specific PK and PD parameters, respectively. Generating random samples from the joint posterior distribution allows the marginal distribution of each parameter to be completely characterized. More details on Bayesian methods in general may be found in (Lunn, Best, Thomas, Wakefield, & Spiegelhalter, 2002).

The model was fit using Bayesian MCMC methods and trace plots were utilized to determine how long to run the burn-in phase and how many samples from the posterior distribution to generate. For these models, a burn-in of 5000 samples was adequate. Samples of between 10000 and 20000 were generated for providing posterior distribution estimates. A mix of noninformative and informative priors were utilized as discussed below.

The MU referencing technique was utilized in NONMEM (Bauer, 2019). In particular, MU_1 (for example) was set equal to THETA(1). Then a particular model parameter was set equal to EXP(MU_1+ETA(1)). So, THETA represents the population value of the parameter on the log-scale. This usually helps to improve the efficiency (speed of convergence, reduced autocorrelation) of the algorithms (MCMC, but also applies to other “advanced” algorithms available) utilized.

Covariates were examined for the final PK/PD model to identify potential factors affecting the PK/PD of PB2452 and its relationship to TICA and TAM. Covariates were also examined for parameters relating uncomplexed TICA and TAM to PRU/LTA/VASP. The covariates considered include demographics (Age, Weight, Gender, BMI, Race), liver functions tests (AST, ALT, Alk. Phos.), baseline PRU/VASP/LTA, kidney marker (eGFR), and hematocrit.

Development of the PK Model:

A diagram of the model considered is in FIG. 1. Ticagrelor is dosed orally and passes through 2 transit compartments prior to entering the central compartment. Ticagrelor is metabolized to its active metabolite TAM, and both ticagrelor and TAM diffuse into peripheral compartments. PB2452 is dosed IV, and directly enters the central compartment. PB2452 can also diffuse into a peripheral compartment. PB2452 binds to ticagrelor and TAM, forming the PB2452-Tica and PB2452-TAM complexes respectively. These complexes render Ticagrelor and TAM inactive. In the model, it is assumed that the complexes are cleared at the same rate as PB2452. Additional compartments for the complex to enter where TICA and TAM return to circulation while PB2452 is removed from the system is also included in the model.

PD Model:

The population PK/PD model relates the model predicted PK of uncomplexed TICA and TAM to the PD measures through Emax models. TICA and TAM were included separately in the model as

$$PRU = Base * ( 1 - \frac{E {max}_{1} * {TICA}^{γ}}{EC {50}_{1}^{γ} + {TICA}^{γ}} - \frac{E {max}_{2} * {TAM}^{γ}}{EC {50}_{2}^{γ} + {TAM}^{γ}} )$$

where Base refers to the baseline (prior to administration of Ticagrelor) value. Emaxᵢ, EC50ᵢ, and the Hill coefficient (γ) are parameters to be estimated. The same structural model was used for PRU, VASP, and LTA. The complexes PB2452-Tica and PB2452-TAM are considered to render Ticagrelor and TAM inactive, and therefore these complexes do not contribute to any PD effects.

Results

A total of 61 (48 Treated with PB2452, 13 Placebo) patients who received PB2452 and/or TICA were included in the PK/PD analysis.

The model in FIG. 1 was based on the premise that PB2452 formed a complex with TICA and TAM that was then presumably removed largely through the kidney. The expected result would be an increase in PRI/LTA/VASP to baseline (pre-ticagrelor administration) levels. Baseline PRU/LTA/VASP were added in the models observed value from the data for each subject. An ETA term was added to allow for potential measurement error. Since ticagrelor has been studied and modeled when administered alone, the PK parameters associated with uncomplexed ticagrelor (TICA) and TAM (and not associated directly with their relationship to PB2452) were fixed to the model-predicted values of the previous model. Astrand et al. (2019).

Because subjects from Cohorts 1-3 were administered PB2452 alone (no TICA was given), data from these cohorts was used to model the PK of uncomplexed PB2452. A two-compartment model was fit to the concentration of PB2452 data versus time. The PK parameters associated with this model are in Table 2. PB2452 has a clearance of 1.88 L/hr and central volume of distribution of 2.86 L. The half-life for the distribution phase and elimination phase are 0.81 hr and 6.68 hr respectively. In the PK/PD model, the parameters associated with the PK of PB2452 (that are not associated with the relationship between PB2452/TICA/TAM) were fixed to these values.

The Emax parameter relating TICA to PRU (as well as VASP and LTA) was fixed to 90%. The EC50 value was estimated to be large, especially relative to the concentrations expected for TICA, suggesting that the Emax would not be reliably estimated using the current data. It is anticipated that this parameter value would be large¹³ so it was fixed at a large value. Kd (affinity of PB2452 for TICA and TAM) was fixed to EXP(−4)=0.018. This value was chosen based on literature^{9, 14}. Finally, fm was set equal to 0.30. This corresponds to about 23% metabolism for TICA to TAM (when no PB2452 is present), which is consistent with literature¹³.

For parameters that were fixed, ETAs were still added to some of them with the OMEGA set to a small value (5%-10%) to allow for some exploration of potential patterns and trends, especially related to covariates. The choice of which parameters to add an ETA to was based on literature¹³. Table 3. presents parameter estimates for the final (covariates included) model, but also indicates which parameters had an ETA added with a small fixed OMEGA value.

For parameters that were to be estimated, most of the prior distributions were uninformative or weakly informative. The prior distribution for all THETAs in the model (natural log of model parameters) was normal with mean=0 and variance=25 except for the THETA corresponding to the EC50 for the effect of TICA on PRU/LTA/VASP and the THETA corresponding to the Emax for the effect of TAM on PRU/LTA/VASP. For the EC50 relating TICA to the PD measures, the natural log of the parameter had a normal prior with mean=4 and variance=100 since that parameter seemed to be large based on initial model runs. For the Emax relating TAM to the PD measures, the natural log of the parameter had a normal prior with mean=—0.10 and variance=0.25 since the value was expected for Emax was expected to be around 90-99 percent.

The hill coefficients for the Emax models relating the metabolism of TICA to PB2452 concentrations and relating TICA and TAM to PRU/LTA/VASP were all set equal to 2. This provided a slight improvement in model fits versus leaving the hill coefficients set to 1.

For interindividual variability, the exponential error structure was used. For residual variability, proportional and additive were utilized. Proportional was used for uncomplexed PB2452 and Total TAM. Proportional plus additive was used for Total PB2452 and Total TICA. Additive was used for PRU/LTA/VASP. When proportional alone was used for Total PB2452 and Total TICA, the estimate for the proportional error was excessively large, leading to simulations that were not meaningful. The problem was mitigated by adding the additive error term.

Based on the covariate plots (ETAs vs covariates) during the initial stages of model development, it appears that the clearance of TAM has a strong relationship with weight. The clearance of TAM (the population value or THETA) was estimated instead of being fixed.

A final base model was run with the metabolism of TICA as a function of the concentration of PB2452 and with the population value (THETA) of clearance for TAM being estimated.

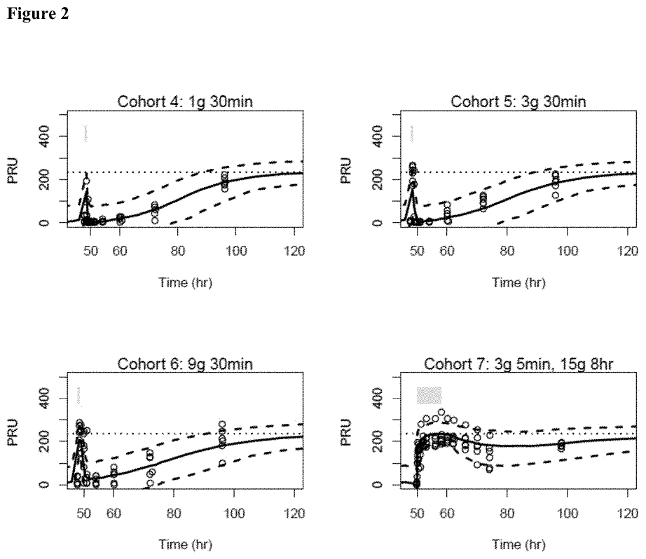

As the study progressed through sequential dose cohorts, it became apparent that some aspects of the PB2452 to TICA and TAM relationships were unaccounted for in the base model. Emerging data suggested that there is potentially some sequestering and recycling of TICA and/or TAM, resulting in these analytes returning to circulation and having an effect on PRU/LTA/VASP. As seen in FIG. 2, where the data, along with simulations from the final model for cohorts 4 through 10 are presented (cohorts 1-3 were administered PB2452 without TICA). In cohorts 4, 5, and 6 the drug had an early effect (the PRU values were increased close to values before TICA was administered) that lasted less than two hours before the PRU values decreased back to the levels that were seen after TICA administration and before PB2452 administration. This was anticipated for cohorts 4 and 5 but was not expected for cohort 6 (9 g PB2452 for 30 minutes) based on initial modeling assumptions. It was anticipated that administration of 9 g PB2452 for 30 minutes would be sufficient to increase PRU values to the pre-TICA baseline and sustain this increase.

Due to the rapid decrease in PRU following PB2452 administration in cohorts 4 through 6, prolonged infusion regimens of PB2452 were investigated in subsequent cohorts. For cohort 7, a bolus plus prolonged infusion was administered with the aim to provide a more sustained effect. The results from this cohort, and those that followed, revealed that a higher dose and prolonged infusion prolonged the increase in PRU. However, an unexpected loss of effect was observed post-cessation of the drug infusion despite dose escalation.

To achieve improved fit to the study data, multiple adjustments were made to the model. The late loss of effect may be due to recycling of ticagrelor and TAM upon elimination of PB2452, so new PB2452-ticagrelor and PB2452-TAM compartments were added to the model which assumes that the complexes move through these new compartments at a rate of Ktr, and that some amount of the ticagrelor and TAM eventually return to circulation as PB2452 is removed from the system. Also, rather than initially summing ticagrelor and TAM as had been performed in other models, these two analytes were modeled separately against PRU/LTA/VASP. These updates led to significant improvements in the fit of the model to the data. To address the underestimation of TAM values, the model was further modified such that metabolism of ticagrelor to TAM was considered a function of the concentration of PB2452. This also led to substantial improvements in the fit of the model to the TAM values.

The final step in model development was addition of significant covariates. Based on examining the covariate plots, weight appeared to be a potentially strong covariate for ETA4 (corresponding to Ktr), ETA8 (corresponding to EC50 relating TAM to PRU/LTA/VASP), and ETA10 (corresponding to the clearance of TAM). Weight was added as a covariate for these terms and was found to be statistically significant for all three. Based on the covariate plots for the final model with the covariates included, no other covariates were added.

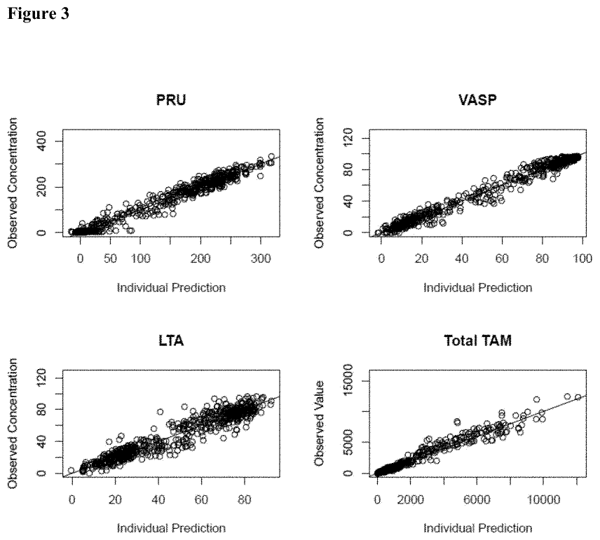

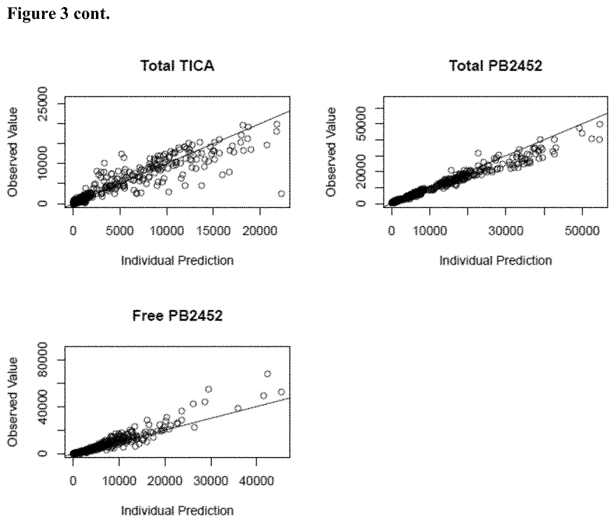

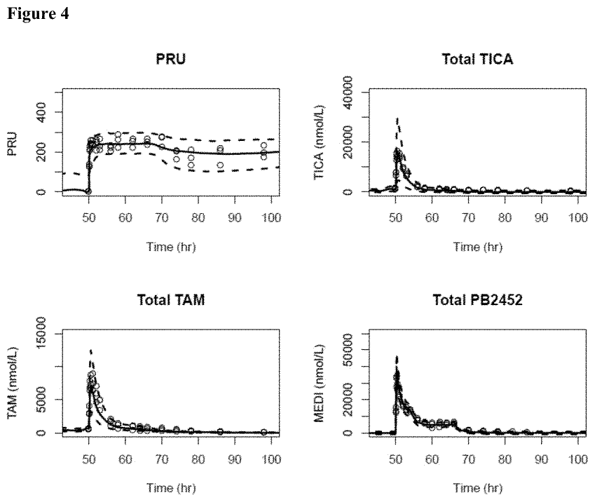

For the final modeling, diagnostic plots were run and appear to be reasonable for all components of the model, including PRU/LTA/VASP, Total TICA, Total TAM, Total PB2452, and uncomplexed PB2452 (FIG. 3). The model slightly overpredicted the Total PB2452 and underpredicted the uncomplexed PB2452. However, the individual plots suggested that the predictions are close to the observed values. Visual predictive checks (simulations from the model for PRU with the data overlaid for the cohorts where TICA and PB2452 were both administered) were then performed and showed that he results for LTA and VASP were similar (FIG. 2). Additionally, the checks indicated that the model was highly predictive of the PRU data from the current study because the median from the simulations (solid lines) typically passed through the middle of the data, and most of the data were contained within the 5^{th} and 95^{th} percentiles (dashed lines). FIG. 4 shows simulations from the model with data overlaid for PRU, total TICA, total TAM, total PB2452, and uncomplexed PB2452 for the cohort where PB2452 was dosed 6 g for 15 minutes followed by 6 g for 4 hours followed by 6 g for 12 hours. This figure shows that the model performed well with any component of the model where observed data was available.

For the final model, different sets of starting values were used for the Bayesian algorithm. Trace plots were produced to examine autocorrelation and convergence. For all THETAs, the model seemed to converge rapidly. THETA1 seemed to exhibit a high degree of autocorrelation. This is likely attributed to the value being arbitrarily large and the model not being sensitive to the value of the parameter. All other THETAs seemed to have a much lower degree of autocorrelation. To minimize any impact of autocorrelation, many posterior samples were generated. A burn-in of 5000 was used per chain followed by generating a sample of 20000 per chain.

The parameter estimates for the final population PK/PD model (PRU) are in Table 3. The parameter estimates for the population PK/PD model for LTA and VASP are similar.

To assess the potential impact of weight in the model, simulations were run using weights of 40 kg, 75 kg, and 110 kg for a dosing schedule of PB2452 of 6 g for 10 minutes (bolus) followed by 6 g for 4 hours and another 6 g for 12 hours. The simulations suggest that the heavier subjects may experience slightly more of a rebound at the later timepoints, but the difference is minor.

Discussion/Conclusion

A phase 1 trial was conducted to evaluate the safety, efficacy, and pharmacokinetic profiles of PB2452 in healthy volunteers 18 to 50 years of age who were pretreated with ticagrelor. The data from this trial was used to develop a semi-mechanistic population PK/PD model. Overall, the model appears to capture the key patterns observed in the data over time and supports use of a standard 3-phase infusion regimen in later phase studies. The model fits the data well based on diagnostic plots, including visual predictive checks. Simulations for the cohorts tested produced results that mirror the observed data.

Given the complex relationship between PB2452 and TICA/TAM, it was important to refine the model to be predictive of the standard 18 gram dosing regimen for PB2452 for subsequent trials. The model supports a dosing regimen initiated with an initial bolus (or a very short infusion) dose to provide immediate reversal, followed by a higher rate infusion (loading regimen) and then a slower maintenance rate infusion to maintain the values of PRU/LTA/VASP necessary for the intended patient populations. Based on model predictions, a 6 gram (g) IV bolus followed by 6 g infused over 4 h and then 6 g over 12 h was identified and tested in study subjects and shown to provide complete reversal within 5 minutes of infusion onset that was sustained for 20-24 h (FIG. 4).

The model and results suggest that PB2452 may have an impact on the metabolism of TICA to TAM. This may be due to PB2452-TICA complex being formed and some of this TICA being metabolized to TAM. The model without this component underestimated the TAM values substantially. It also shows that as PB2452 binds to TICA and TAM, more TICA and TAM may rapidly redistribute to the central compartment from the periphery, causing a rapid return of PRU/LTA/VASP to levels observed prior to PB2452 administration. The apparent sequestering and partial recycling of TICA and TAM evident in later timepoints post-PB2452 infusion may be due to post-glomerular reabsorption of PB2452-TICA and -TAM complexes into tubular cells, lysosomal degradation of PB2452, and recycling of TICA and TAM back into circulation. This possibility for antibody fragments is discussed in the literature¹⁵. The addition of extra PB2452-TICA and PB2452-TAM complex compartments allows for this to be represented in the model. They are not necessarily physiologic compartments but may depict a process of complex dissociation and TICA (or TAM) return to circulation. Some of is the recirculated TICA may be metabolized to TAM could explain the observed relationship between TAM concentration and PB2452 concentrations.

The EC50 for TICA was large (greater than 20000 nmol/L) which is well beyond the range of uncomplexed TICA expected based on the model (less than 2000 nmol/L). This suggests that the effect of TICA may be closer to a gradually increasing log linear model. The EC50 for TAM is much lower at 98.5 nmol/L. The model presented here is different from others in literature^{13, 16} where the relationship is either modeled as a sum of TICA and TAM or just TICA alone. The model presented here fits the placebo data (subjects who received TICA plus a placebo) well, suggesting that the model here performs as well as those used elsewhere. It is possible that in the previous studies, the natural relationship between TICA and TAM caused it to be difficult to distinguish the contribution due to each, whereas in the present study with PB2452 added, this relationship is altered.

Since complexes of PB2452 with TICA and TAM are expected to be removed in the kidney, either by degradation or urinary elimination, eGFR was explored as covariate. However, no effects with eGFR were observed perhaps due to the healthy study population with most eGFR values within a normal range (74.75 to 162.80 mL/min/1.73 m² calculated by the abbreviated MDRD equation).

In conclusion, we developed a semi-mechanistic model to explain the relationships between PB2452, TICA, TAM, and the effects on PRU/LTA/VASP. The model explained the data well and assisted with finding a dose and administration regimen subsequent clinical trials. The model may be useful to help guide development of PB2452 as an antidote for ticagrelor in hospitalized patients. Updating the model with patient data may then help predict whether dose adjustments may be needed in different subpopulations, e.g. elderly, different ethnicities, as well.

TABLE 1
Dosing Regimen for Each Cohort

| | Pre-PB2452 | | | |
|---|---|---|---|---|
| | ticagrelor dosing | PB2452 | PB2452 | N |
| Cohort | to steady state | dose (g) | Infusion Time | (Active:Placebo) |
| 1 | | 0.1 | 30 minutes | 3:1 |
| 2 | | 0.3 | 30 minutes | 3:1 |
| 3 | | 1.0 | 30 minutes | 3:1 |
| 4^{a} | 180 mg + | 1.0 | 30 minutes | 6:2 |
| | 90 mg bid | | | |
| 5^{a} | 180 mg + | 3.0 | 30 minutes | 6:2 |
| | 90 mg bid | | | |
| 6^{a} | 180 mg + | 9.0 | 30 minutes | 6:2 |
| | 90 mg bid | | | |
| 7^{b} | 180 mg + | 18.0 | 3 g 5 min + | 6:2 |
| | 90 mg bid | | 15 g 8 hr | |
| 8^{b} | 180 mg + | 18.0 | 6 g 15 min + | 6:2 |
| | 90 mg bid | | 6 g 3 hr + | |
| | | | 6 g 8 hr 45 min | |
| 9^{b} | 180 mg + | 18.0 | 6 g 15 min + | 3:1 |
| | 90 mg bid | | 6 g 4 hr + | |
| | | | 6 g 12 hr | |
| 10^{b} | 180 mg + | 18.0 | 6 g 10 min + | 6:2 |
| | 90 mg bid | | 6 g 3 hr + | |
| | | | 6 g 13 hr | |

Note:
^{a}The last dose of ticagrelor and PB2452 were administered simultaneously
^{b}The last dose of ticagrelor was give two hours prior to the administration of PB2452.

TABLE 2
Population PK Parameters of Final PK Model (PB2452 alone)

| Parameters | Final | 95% CI | | Inter-individual |
|---|---|---|---|---|
| (Units) | Estimate | Lower | Upper | Variability |

CL (L/hr) = EXP(THETA1)

| | | | | |
|---|---|---|---|---|
| THETA1 | 0.632 | 0.383 | 0.881 | 37.8% |

V1 (L) = EXP(THETA2)

| | | | | |
|---|---|---|---|---|
| THETA2 | 1.05 | 0.781 | 1.32 | 40.4% |

Q (L/hr) = EXP(THETA3)

| | | | | |
|---|---|---|---|---|
| THETA3 | −0.770 | −1.07 | −0.470 | 42.8% |

V2 (L) = EXP(THETA4)

| | | | | |
|---|---|---|---|---|
| THETA4 | 1.24 | 0.744 | 1.74 | 62.9% |

Residual Variability: CV = 7.11%

TABLE 3
Population PK/PD Parameters of Final PK/PD Model

| | | | | Inter-individual |
|---|---|---|---|---|
| Parameters | Final | 95% CI | | Variability |
| (Units) | Estimate | Lower | Upper | (CV %) |

EC50 (nmol/L) = EXP(THETA1) EC50 for relating TICA to PRU

| | | | | |
|---|---|---|---|---|
| THETA1 | 10.6 | 8.76 | 12.4 | 23.3% |

Emax = EXP(−0.1) Emax for relating TICA to PRU

Fixed in model

Kon (nmol⁻¹ × hr⁻¹) = EXP(THETA2)

| | | | | |
|---|---|---|---|---|
| THETA2 | −5.56 | −5.73 | −5.39 | 43.6% |

Kd (nmol) = EXP(−4)

Fixed in model

Kd2 (nmol) = EXP(THETA3)

| | | | | |
|---|---|---|---|---|
| THETA3 | 2.04 | 1.86 | 2.22 | 25.9% |

Ktr (hr⁻¹) = EXP(THETA4 + THETA13*(LOG(WT) − 4.35))

| | | | | |
|---|---|---|---|---|
| THETA4 | −1.22 | −1.33 | −1.11 | 25.0% |
| THETA13 | 1.46 | 0.841 | 2.08 | |

Kon2 (nmol⁻¹ × hr⁻¹) = EXP(THETA5)

| | | | | |
|---|---|---|---|---|
| THETA5 | −3.74 | −3.91 | −3.57 | 30.4% |

Emaxf = EXP(THETA6) Emax for relating
metabolism of TICA to PB2452 concentrations

| | | | | |
|---|---|---|---|---|
| THETA6 | 2.98 | 2.76 | 3.20 | 23.6% |

ECf (nmol/L) = EXP(THETA7) EC50 for relating
metabolism of TICA to PB2452 concentrations

| | | | | |
|---|---|---|---|---|
| THETA7 | 9.36 | 9.10 | 9.62 | 59.8% |

EC502 (nmol/L) = EXP(THETA8 +
THETA12*(LOG(WT) − 4.35)) EC50 for relating TAM to PRU

| | | | | |
|---|---|---|---|---|
| THETA8 | 4.59 | 4.49 | 4.69 | 25.3% |
| THETA12 | −0.965 | −1.49 | −0.442 | |

Emax2 = EXP(THETA9) Emax for relating TAM to PRU

| | | | | |
|---|---|---|---|---|
| THETA9 | 0.0181 | −0.0421 | 0.0783 | 20.7% |

KA = EXP(2.3) Absorption parameter for TICA

| | |
|---|---|
| Fixed in model | 10% Fixed |

CL/F (L/hr) = EXP(2.81) Clearance of TICA

| | |
|---|---|
| Fixed in model | 10% Fixed |

V1/F (L) = EXP(5.04) Central Volume of TICA

Fixed in model

V2/F (L) = EXP(4.02) Peripheral Volume of TICA

Fixed in model

Q1/F L (hr) = EXP(2.34) Intercompartmental Clearance of TICA

| | |
|---|---|
| Fixed in model | 10% Fixed |

CLM (L/hr) = EXP(THETA11 + THETA10*(LOG(WT) − 4.35)) Clearance of TAM

| | | | | |
|---|---|---|---|---|
| THETA10 | 1.31 | 0.912 | 1.71 | 23.9% |
| THETA11 | 1.93 | 1.86 | 2.00 | |

VM1 (L) = EXP(1.95) Central volume of TAM

| | |
|---|---|
| Fixed in model | 10% Fixed |

VM2 (L) = EXP(3.74) Peripheral volume of TAM

Fixed in model

Q2M (L/hr) = EXP(1.48) Intercompartment Clearance of TAM

| | |
|---|---|
| Fixed in model | 5% Fixed |

CL_ant (L/hr) = EXP(0.631) Clearance of PB2452

Fixed in model

Q_ant (L/hr) = EXP(−0.765) Intercompartmental Clearance of PB2452

| | |
|---|---|
| Fixed in model | 5% Fixed |

V_ant (L) = EXP(1.05) Central Volume of PB2452

| | |
|---|---|
| Fixed in model | 5% Fixed |

V_ant_perp (L) = EXP(1.28) Peripheral Volume of PB2452

| | |
|---|---|
| Fixed in model | 5% Fixed |

Base = Log(BPRU) Baseline PRU

| | |
|---|---|
| Fixed in model | 10% Fixed |
| to observed | |
| baseline values. | |

fm = 0.3 Metabolisim rate of TICA is fm*Elimination rate of TICA.
This is the value for metabolism when there is no PB2452 present.

Fixed in model

Koff = Kon*Kd
Koff2 = Kon*Kd2
Residual Variability:
PRU: Additive SD = 20.3
Uncomplexed PB2452: 28.2%
Total PB2452: 13.7%
Additive SD = 504
Total TICA: 42.4%
Additive SD = 332
Total TAM: 23.1%

1. A method of modeling, simulating, and/or determining an effective dosing regimen of an antibody or fragment thereof that binds an inhibitor of P2Y₁₂ receptor signaling or P2Y12 receptor-induced platelet aggregation in a patient population, the method comprising:
a) determining a pharmacokinetic-pharmacodynamic (PD/PD) model that characterizes the relationship between ticagrelor and ticagrelor active metabolite (TAM) individually versus P2Y₁₂ receptor-induced platelet aggregation and P2Y₁₂ receptor signaling;
b) wherein if the determination in (a) indicates that the P2Y₁₂ receptor-induced platelet aggregation and P2Y₁₂ receptor signaling is decreasing, the predicted effective dosing regimen is a higher dose of the antibody or fragment thereof infused at a faster rate or over a longer period of time; or
c) wherein if the determination in (a) indicates that the P2Y₁₂ receptor-induced platelet aggregation and P2Y₁₂ receptor signaling is increasing, the predicted effective dosing regimen is a lower dose of the antibody or fragment thereof and/or infused at a slower rate or over a shorter period of time.

2. The method of claim 1, wherein the dosing regimen is sufficient to increase P2Y₁₂ receptor-induced platelet aggregation and P2Y₁₂ receptor signaling values towards the baseline observed before administration of the inhibitor of the P2Y₁₂ receptor signaling.

3. The method of claim 2, wherein the dosing regimen is effective to sustain the increase of P2Y₁₂ receptor-induced platelet aggregation and P2Y₁₂ receptor signaling.

4. The method of claim 1, wherein P2Y₁₂ receptor-induced platelet aggregation and/or P2Y₁₂ receptor signaling is determined by one or more methods selected from light transmittance aggregometry (LTA), VerifyNow™-based P2Y₁₂ reactivity units (PRU), vasodilatory stimulated phosphoprotein (VASP) phosphorylation, and/or other platelet-function or P2Y₁₂-receptor-signaling assays.

5. The method of claim 1, wherein the metabolism of ticagrelor to TAM is modeled as a function of the concentration values of the antibody or fragment thereof.

6. The method of claim 1, wherein the pharmacokinetic-pharmacodynamic (PK/PD) model that characterizes the relationship between ticagrelor and ticagrelor active metabolite (TAM) individually versus P2Y₁₂ receptor-induced platelet aggregation and P2Y12 receptor signaling is determined using the following equation or the same structural model used additionally VASP, and LTA:
$$PRU = Base * ( 1 - \frac{E {max}_{1} * {TICA}^{γ}}{EC {50}_{1}^{γ} + {TICA}^{γ}} - \frac{E {max}_{2} * {TAM}^{γ}}{EC {50}_{2}^{γ} + {TAM}^{γ}} )$$

7. The method of claim 1, wherein the predicted effective dosing regimen comprises an initial bolus followed by a higher rate infusion, and then followed by a slower rate infusion.

8. The method of claim 1, wherein the values of P2Y₁₂ receptor-induced platelet aggregation and P2Y12 receptor signaling necessary for the intended patient population are maintained.

9. The method of claim 8, wherein the P2Y₁₂ receptor-induced platelet aggregation and P2Y₁₂ receptor signaling levels are maintained for about 1 to 48 hours.

10. The method of claim 9, wherein the P2Y₁₂ receptor-induced platelet aggregation and P2Y₁₂ receptor signaling levels are maintained for about 10-30 hours.

11. (canceled)

12. The method of claim 1, wherein the dosing regimen provides complete reversal of the inhibitor of a P2Y₁₂ receptor-induced platelet aggregation and P2Y₁₂ receptor signaling.

13. The method of claim 1, wherein the dosing regimen provides complete reversal of the inhibitor of a P2Y₁₂ receptor-induced platelet aggregation and P2Y₁₂ receptor signaling within about 5 minutes of infusion onset.

14. The method of claim 13, wherein the complete reversal is sustained for at least 20 to 24 hours.

15. The method of claim 1, wherein administration of the antibody or fragment thereof restores platelet function.

16. The method of claim 15, wherein administration of the antibody or fragment thereof restores platelet aggregation or platelet receptor signaling.

17. The method of claim 16, wherein administration of the antibody or fragment thereof restores platelet aggregation or platelet receptor signaling to at least 80% of baseline.

18. The method of claim 16, wherein administration of the antibody or fragment thereof restores platelet aggregation or platelet receptor signaling within 1 minute to 60 minutes of administration.

19. (canceled)

20. The method of claim 16, wherein administration of the antibody or fragment thereof provides a sustained restoration of platelet aggregation or platelet receptor signaling.

21. The method of claim 20, wherein the restoration of platelet aggregation or platelet receptor signaling is sustained for at least 12 hours after administration.

22-23. (canceled)

24. The method of claim 1, wherein the antibody or fragment thereof is a Fab and the patient is administered a dose between about 1 g and about 48 g.

25-57. (canceled)


SEQUENCE LISTING (86 sequences)

SEQ ID NO: 1 - DNA, 363 bp, Homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcaacagagtacgacctgcaacggcctttcgggtttgacttctggggcaaggggacaatggtcaccgtctcgagt

SEQ ID NO: 2 - protein, 121 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCATEYDLQRPFGFDFWGKGTMVTVSS

SEQ ID NO: 3 - protein, 5 aa, Homo sapiens
SYAMS

SEQ ID NO: 4 - protein, 17 aa, Homo sapiens
AISGSGGSTYYADSVKG

SEQ ID NO: 5 - protein, 12 aa, Homo sapiens
EYDLQRPFGFDF

SEQ ID NO: 6 - DNA, 333 bp, Homo sapiens
tcctatgtgctgactcagccaccctcagcgtctggggcccccgggcagagggctaccatctcctgctctggaagcagctccaacatcggaagtaatcttgtgaactggtaccaacaattcccaggagaggcccccaagctcctcatctttagtgacaatcaacgaccctcaggggtccctgaccgattctctggctccaggtctggcacctcagcctccctggccatcagtgggctccagtccgaggatgaggctgattattactgtgcaacgtgggatgacagactggatggttatgtggtattcggcggagggaccaagctgaccgtccta

SEQ ID NO: 7 - protein, 111 aa, Homo sapiens
SYVLTQPPSASGAPGQRATISCSGSSSNIGSNLVNWYQQFPGEAPKLLIFSDNQRPSGVPDRFSGSRSGTSASLAISGLQSEDEADYYCATWDDRLDGYVVFGGGTKLTVL

SEQ ID NO: 8 - protein, 13 aa, Homo sapiens
SGSSSNIGSNLVN

SEQ ID NO: 9 - protein, 7 aa, Homo sapiens
SDNQRPS

SEQ ID NO: 10 - protein, 12 aa, Homo sapiens
ATWDDRLDGYVV

SEQ ID NO: 11 - DNA, 345 bp, Homo sapiens
caggtacagctgcagcagtcaggggctgaggtgaagaagcctggggcctcagtgaaggtttcctgcaaggcttctggatacaccttcattacctatggtattcactgggtgcgccaggcccccggacaagggcttgagtggatgggatggatcgaccccgggcatggttacacaaaatattcacagaagttccagggcagagtcaccattaccagggacacatccgcgagcacagcctacatggagatgagcagcctcagatctgaagacacggctgtgtattactgtgcgagagcggacctgggtgactactggggccggggaaccctggtcaccgtctcgagt

SEQ ID NO: 12 - protein, 115 aa, Homo sapiens
QVQLQQSGAEVKKPGASVKVSCKASGYTFITYGIHWVRQAPGQGLEWMGWIDPGHGYTKYSQKFQGRVTITRDTSASTAYMEMSSLRSEDTAVYYCARADLGDYWGRGTLVTVSS

SEQ ID NO: 13 - protein, 5 aa, Homo sapiens
TYGIH

SEQ ID NO: 14 - protein, 17 aa, Homo sapiens
WIDPGHGYTKYSQKFQG

SEQ ID NO: 15 - protein, 6 aa, Homo sapiens
ADLGDY

SEQ ID NO: 16 - DNA, 330 bp, Homo sapiens
cagtctgtcgtgacgcagccgccctcagtgtctgcggccccaggacagaaggtcaccatctcctgctctggaagcagctccaacattgggaagaattatgtttcctggttccagcagctcccaggtacagcccccaaactcctcatttatgacaatcataagcgaccctcagggattcctgaccgattctctgcctccaagtctggcacgtcagccaccctggtcatctccggtctccagactggggacgaggcccattattactgcggaacatgggataccagactgagtgctggggtgttcggcggagggaccaaggtcaccgtccta

SEQ ID NO: 17 - protein, 110 aa, Homo sapiens
QSVVTQPPSVSAAPGQKVTISCSGSSSNIGKNYVSWFQQLPGTAPKLLIYDNHKRPSGIPDRFSASKSGTSATLVISGLQTGDEAHYYCGTWDTRLSAGVFGGGTKVTVL

SEQ ID NO: 18 - protein, 13 aa, Homo sapiens
SGSSSNIGKNYVS

SEQ ID NO: 19 - protein, 7 aa, Homo sapiens
DNHKRPS

SEQ ID NO: 20 - protein, 11 aa, Homo sapiens
GTWDTRLSAGV

SEQ ID NO: 21 - DNA, 360 bp, Homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtggccatgatagtagtggttactcctactcctttgacttctgggggcgggggaccacggtcaccgtctcgagt

SEQ ID NO: 22 - protein, 120 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCGHDSSGYSYSFDFWGRGTTVTVSS

SEQ ID NO: 23 - protein, 5 aa, Homo sapiens
SYAMS

SEQ ID NO: 24 - protein, 17 aa, Homo sapiens
AISGSGGSTYYADSVKG

SEQ ID NO: 25 - protein, 11 aa, Homo sapiens
DSSGYSYSFDF

SEQ ID NO: 26 - DNA, 330 bp, Homo sapiens
cagtctgtgttgacgcagccgccctcagcgtctgggacccccgggcagagggtcaccatctcttgttctggcaacatctccaacatcggaagtaacactgtcaactggtatcaacacgtcccaggagcggcccccagactcctcatctatgttaatgatcagcggccgtcaggggtccctgaccgattctctggctccaagtctggcacctcagcctccctggccatcagtgggctccagtctgaagatgaggctgattattactgtgcaacgtgggatgacaccctgaatggaggggtcttcggcggagggaccaagctgaccgtccta

SEQ ID NO: 27 - protein, 110 aa, Homo sapiens
QSVLTQPPSASGTPGQRVTISCSGNISNIGSNTVNWYQHVPGAAPRLLIYVNDQRPSGVPDRFSGSKSGTSASLAISGLQSEDEADYYCATWDDTLNGGVFGGGTKLTVL

SEQ ID NO: 28 - protein, 13 aa, Homo sapiens
SGNISNIGSNTVN

SEQ ID NO: 29 - protein, 7 aa, Homo sapiens
VNDQRPS

SEQ ID NO: 30 - protein, 11 aa, Homo sapiens
ATWDDTLNGGV

SEQ ID NO: 31 - DNA, 387 bp, Homo sapiens
caggtgcagctgcaggagtccggggctgaggtgaagaagcctgggtcctcggtgagggtctcctgcaaggcttctggaggcaccttcgacagttatagtatccattgggtgcgccaggcccctggacaagggcttgagtggatgggagggatcatccctgcctttgggacattaagcagcgcacaggacttccaggccagagtcaccattagcgcggacaagtccacgagcacagcctatatggagctgagcggcctgagatctgaggacacggccgtatattactgtgcgagagggtcccatctttacgatttttggagtgcttctcatccccccaatgatgctcttgctatttggggccaaggaaccctggtcaccgtctcgagt

SEQ ID NO: 32 - protein, 129 aa, Homo sapiens
QVQLQESGAEVKKPGSSVRVSCKASGGTFDSYSIHWVRQAPGQGLEWMGGIIPAFGTLSSAQDFQARVTISADKSTSTAYMELSGLRSEDTAVYYCARGSHLYDFWSASHPPNDALAIWGQGTLVTVSS

SEQ ID NO: 33 - protein, 5 aa, Homo sapiens
SYSIH

SEQ ID NO: 34 - protein, 17 aa, Homo sapiens
GIIPAFGTLSSAQDFQA

SEQ ID NO: 35 - protein, 20 aa, Homo sapiens
GSHLYDFWSASHPPNDALAI

SEQ ID NO: 36 - DNA, 330 bp, Homo sapiens
cagtctgtcgtgacgcagccgccctcagtgtctgcggccccaggacagaaggtcaccatctcctgctctggaagcaactccgacattggcaacaattatgtgtcgtggtaccaacagctcccaggaacagcccccaaactcctcatttatgacaataataaacgaccctcagggattcctgaccgattctctggctccaagtctggcacgtcagccaccctggccatcaccggactccaggctggggacgaggccgattattactgcgggacatgggatatcagcctgagcgctggcttgttcggcggagggaccaaggtcaccgtccta

SEQ ID NO: 37 - protein, 110 aa, Homo sapiens
QSVVTQPPSVSAAPGQKVTISCSGSNSDIGNNYVSWYQQLPGTAPKLLIYDNNKRPSGIPDRFSGSKSGTSATLAITGLQAGDEADYYCGTWDISLSAGLFGGGTKVTVL

SEQ ID NO: 38 - protein, 13 aa, Homo sapiens
SGSNSDIGNNYVS

SEQ ID NO: 39 - protein, 7 aa, Homo sapiens
DNNKRPS

SEQ ID NO: 40 - protein, 11 aa, Homo sapiens
GTWDISLSAGL

SEQ ID NO: 41 - DNA, 387 bp, Homo sapiens
caggtgcagctgcaggagtccggggctgaggtgaagaagcctgggtcctcggtgagggtctcctgcaaggcttctggaggcaccttcgacagttatagtatccattgggtgcgccaggcccctggacaagggcttgagtggatgggagggatcatccctgcctttgggacattaagcagcgcacaggacttccaggccagagtcaccattagcgcggacaagtccacgagcacagcctatatggagctgagcggcctgagatctgaggacacggccgtatattactgtgcgagagggagcttcgactacaggttttggagtgcttctcatccccccaatgatgctcttgctatttggggccaaggaaccctggtcaccgtctcgagt

SEQ ID NO: 42 - protein, 129 aa, Homo sapiens
QVQLQESGAEVKKPGSSVRVSCKASGGTFDSYSIHWVRQAPGQGLEWMGGIIPAFGTLSSAQDFQARVTISADKSTSTAYMELSGLRSEDTAVYYCARGSFDYRFWSASHPPNDALAIWGQGTLVTVSS

SEQ ID NO: 43 - protein, 5 aa, Homo sapiens
SYSIH

SEQ ID NO: 44 - protein, 17 aa, Homo sapiens
GIIPAFGTLSSAQDFQA

SEQ ID NO: 45 - protein, 20 aa, Homo sapiens
GSFDYRFWSASHPPNDALAI

SEQ ID NO: 46 - DNA, 330 bp, Homo sapiens; identical to SEQ ID NO: 36

SEQ ID NO: 47 - protein, 110 aa, Homo sapiens; identical to SEQ ID NO: 37

SEQ ID NO: 48 - protein, 13 aa, Homo sapiens
SGSNSDIGNNYVS

SEQ ID NO: 49 - protein, 7 aa, Homo sapiens
DNNKRPS

SEQ ID NO: 50 - protein, 11 aa, Homo sapiens
GTWDISLSAGL

SEQ ID NO: 51 - DNA, 387 bp, Homo sapiens
caggtgcagctgcaggagtccggggctgaggtgaagaagcctgggtcctcggtgagggtctcctgcaaggcttctggaggcaccttcgacagttatagtatccattgggtgcgccaggcccctggacaagggcttgagtggatgggagggatcatccctgcctttgggacattaagcagcgcacaggacttccaggccagagtcaccattagcgcggacaagtccacgagcacagcctatatggagctgagcggcctgagatctgaggacacggccgtatattactgtgcgagaggctccttcgactactacttttggagtgcttctcatccccccaatgatgctcttgctatttggggccaaggaaccctggtcaccgtctcgagt

SEQ ID NO: 52 - protein, 129 aa, Homo sapiens
QVQLQESGAEVKKPGSSVRVSCKASGGTFDSYSIHWVRQAPGQGLEWMGGIIPAFGTLSSAQDFQARVTISADKSTSTAYMELSGLRSEDTAVYYCARGSFDYYFWSASHPPNDALAIWGQGTLVTVSS

SEQ ID NO: 53 - protein, 5 aa, Homo sapiens
SYSIH

SEQ ID NO: 54 - protein, 17 aa, Homo sapiens
GIIPAFGTLSSAQDFQA

SEQ ID NO: 55 - protein, 20 aa, Homo sapiens
GSFDYYFWSASHPPNDALAI

SEQ ID NO: 56 - DNA, 330 bp, Homo sapiens; identical to SEQ ID NO: 36

SEQ ID NO: 57 - protein, 110 aa, Homo sapiens; identical to SEQ ID NO: 37

SEQ ID NO: 58 - protein, 13 aa, Homo sapiens
SGSNSDIGNNYVS

SEQ ID NO: 59 - protein, 7 aa, Homo sapiens
DNNKRPS

SEQ ID NO: 60 - protein, 11 aa, Homo sapiens
GTWDISLSAGL

SEQ ID NO: 61 - DNA, 387 bp, Homo sapiens; identical to SEQ ID NO: 31

SEQ ID NO: 62 - protein, 129 aa, Homo sapiens; identical to SEQ ID NO: 32

SEQ ID NO: 63 - protein, 5 aa, Homo sapiens
SYSIH

SEQ ID NO: 64 - protein, 17 aa, Homo sapiens
GIIPAFGTLSSAQDFQA

SEQ ID NO: 65 - protein, 20 aa, Homo sapiens
GSHLYDFWSASHPPNDALAI

SEQ ID NO: 66 - DNA, 330 bp, Homo sapiens
cagtctgtcgtgacgcagccgccctcagtgtctgcggccccaggacagaaggtcaccatctcctgctctggaagcaactccgacattggcaacaattatgtgtcgtggtaccaacagctcccaggaacagcccccaaactcctcatttatgacaataataaacgaccctcagggattcctgaccgattctctggctccaagtctggcacgtcagccaccctggccatcaccggactccaggctggggacgaggccgattattactgcgggacatggctgtacgaccgggccgtcggcttgttcggcggagggaccaaggtcaccgtccta

SEQ ID NO: 67 - protein, 110 aa, Homo sapiens
QSVVTQPPSVSAAPGQKVTISCSGSNSDIGNNYVSWYQQLPGTAPKLLIYDNNKRPSGIPDRFSGSKSGTSATLAITGLQAGDEADYYCGTWLYDRAVGLFGGGTKVTVL

SEQ ID NO: 68 - protein, 13 aa, Homo sapiens
SGSNSDIGNNYVS

SEQ ID NO: 69 - protein, 7 aa, Homo sapiens
DNNKRPS

SEQ ID NO: 70 - protein, 11 aa, Homo sapiens
GTWLYDRAVGL

SEQ ID NO: 71 - DNA, 387 bp, Homo sapiens; identical to SEQ ID NO: 51

SEQ ID NO: 72 - protein, 129 aa, Homo sapiens; identical to SEQ ID NO: 52

SEQ ID NO: 73 - protein, 5 aa, Homo sapiens
SYSIH

SEQ ID NO: 74 - protein, 17 aa, Homo sapiens
GIIPAFGTLSSAQDFQA

SEQ ID NO: 75 - protein, 20 aa, Homo sapiens
GSFDYYFWSASHPPNDALAI

SEQ ID NO: 76 - DNA, 330 bp, Homo sapiens; identical to SEQ ID NO: 66

SEQ ID NO: 77 - protein, 110 aa, Homo sapiens; identical to SEQ ID NO: 67

SEQ ID NO: 78 - protein, 13 aa, Homo sapiens
SGSNSDIGNNYVS

SEQ ID NO: 79 - protein, 7 aa, Homo sapiens
DNNKRPS

SEQ ID NO: 80 - protein, 11 aa, Homo sapiens
GTWLYDRAVGL

SEQ ID NO: 81 - protein, 234 aa, Homo sapiens
QVQLQESGAEVKKPGSSVRVSCKASGGTFDSYSIHWVRQAPGQGLEWMGGIIPAFGTLSSAQDFQARVTISADKSTSTAYMELSGLRSEDTAVYYCARGSFDYYFWSASHPPNDALAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 82 - protein, 216 aa, Homo sapiens
QSVVTQPPSVSAAPGQKVTISCSGSNSDIGNNYVSWYQQLPGTAPKLLIYDNNKRPSGIPDRFSGSKSGTSATLAITGLQAGDEADYYCGTWLYDRAVGLFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 83 - protein, 234 aa, Homo sapiens; identical to SEQ ID NO: 81
  PB2452 VH with CH1

SEQ ID NO: 84 - DNA, 702 bp, Homo sapiens
  cDNA encoding PB2452 VH with CH1
caggttcaattgcaagaaagcggtgcagaagttaaaaaaccgggtagcagcgttcgtgttagctgtaaagcaagcggtggcacctttgatagctatagcattcattgggttcgtcaggcaccgggtcagggtctggaatggatgggtggtattattccggcatttggcaccctgagcagcgcacaggattttcaggcacgtgttaccattagcgcagataaaagcaccagcaccgcatatatggaactgagcggtctgcgtagcgaagataccgcagtgtattattgtgcacgtggtagcttcgattattacttttggagcgcaagccatccgcctaatgatgcactggcaatttggggtcagggcaccctggttaccgttagcagcgcaagcaccaaaggtccgagcgtttttccgctggcaccgagcagcaaaagcaccagtggtggcaccgcagcactgggttgtctggttaaagattattttccggaaccggttaccgtgagctggaatagcggtgcactgaccagcggtgttcatacctttccggcagttctgcagagcagcggtctgtatagcctgagcagcgttgttaccgttccgagcagcagcctgggcacccagacctatatttgtaatgttaatcataaaccgagcaataccaaagtggataaacgtgttgaaccgaaaagctgcgataaa

SEQ ID NO: 85 - protein, 216 aa, Homo sapiens; identical to SEQ ID NO: 82
  PB2452 VL with CL

SEQ ID NO: 86 - DNA, 648 bp, Homo sapiens
  cDNA encoding PB2352 VL with CL
cagagcgttgtgacccagcctccgagcgttagcgcagctccgggtcagaaagtgaccattagctgtagcggtagcaatagcgatattggcaataactatgttagctggtatcagcagctgcctggcaccgcaccgaaactgctgatttatgataataacaaacgtccgagcggtattccggatcgttttagcggtagtaaaagcggcaccagcgcaaccctggcaattaccggtctgcaagccggtgatgaagcagattattattgtggcacctggctgtatgatcgtgcagttggtctgtttggtggtggcaccaaagttaccgttctgggtcagccgaaagcagcaccgagcgttaccctgtttccgcctagcagcgaagaactgcaggcaaataaagcaaccctggtttgtctgatcagcgatttttatccgggtgcagttaccgttgcatggaaagcagatagcagtccggttaaagccggtgttgaaaccaccaccccgagcaaacagagcaataacaaatatgcagcaagcagctatctgagcctgacaccggaacagtggaaaagccatcgtagctatagctgtcaggttacccatgaaggtagcaccgttgaaaaaaccgttgcaccgaccgaatgtagc